(19) United States
(12) Patent Application Publication
(10) Pub. No.: US 2025/0042880 A1
(43) Pub. Date: Feb. 6, 2025

(54) DIAMINOBUTOXY-SUBSTITUTED ISOFLAVONOIDS AS MITOCHONDRIAL COMPLEX I INHIBITORS FOR CANCER TREATMENT

(71) Applicant: UNIVERSITY OF KENTUCKY RESEARCH FOUNDATION, Lexington, KY (US)

(72) Inventors: Chunming Liu, Lexington, KY (US); David Watt, Lexington, KY (US); Xifu Liu, Shjiazhuang, Hebei (CN)

(21) Appl. No.: 18/716,131

(22) PCT Filed: Dec. 6, 2022
(86) PCT No.: PCT/US22/52038

§ 371 Date: Jun. 3, 2024

Related U.S. Application Data
(60) Provisional application No. 63/286,303, filed on Dec. 6, 2021.

(51) Int. Cl. C07D 405/12; A61K 31/352; A61K 31/496; A61P 3/10; A61P 35/00; C07D 311/36

(52) U.S. Cl. CPC C07D 405/12; A61K 31/352; A61K 31/496; A61P 3/10; A61P 35/00; C07D 311/36

(57) ABSTRACT

The presently-disclosed subject matter relates to diaminobutoxy-substituted isoflavonoids and their use thereof as mitochondrial complex I inhibitors for the treatment of cancer.

### RELATED APPLICATIONS

This application claims priority from U.S. Provisional Application Serial No. U.S. 63/286,303, filed Dec. 6, 2021, the entire disclosure of which is incorporated herein by this reference.

### GOVERNMENT INTEREST

This invention was made with government support under grant numbers R01 CA172379 and P30 CA177558 awarded by the National Cancer Institute (NCI). The government has certain rights in the invention.

### TECHNICAL FIELD

The present invention relates to compounds for the treatment of cancer. In particular, the presently-disclosed subject matter relates to diaminobutoxy-substituted isoflavonoids and the use thereof as mitochondrial complex I inhibitors for the treatment of cancer.

### INTRODUCTION

Colorectal cancer (CRC) is the third most common malignancy diagnosed globally and the fourth leading cause of cancer-related deaths worldwide, with approximately 1.8 million new cases per year worldwide. Current projections suggest that the societal burden of CRC will increase by 60% by 2030 (1,2). Despite significant advances in cancer treatments, the five-year survival rate of late-stage CRC (i.e., stage IV or metastatic CRC) is only 14.2% (3,4). Because of the staggering toll this disease has on society and the frightening projections for its increase, new modalities for treating late-stage CRC represent an imperative for cancer research.

Cancer cells undergo a significant level of “metabolic remodeling” to provide sufficient ATP to maintain cell survival and to promote rapid growth (5-7). In the 1920s, Otto Warburg made his seminal observation that tumor cells metabolize more glucose to lactate than that metabolized by normal cells. By directly measuring lactate production and oxygen consumption rates in thin slices of rat liver carcinomas and normal liver tissues, Warburg found that normal liver tissue exhibited a so-called Pasteur effect (i.e., inhibition of lactate production in the presence of oxygen), whereas tumor tissues maintained lactate production regardless of oxygen tension (8,9). This phenomenona that cancer cells preferentially use glycolysis for ATP production even in the presence of oxygen that is needed in oxidative phosphorylation is called the Warburg effect (10). That is, ATP production occurs in the cytoplasm as a result of glycolysis and also occurs in mitochodria as a result of the electron transport chain (ETC) (i.e., complex I) as a result of oxidative phosphorylation. It has recently become the focus of intensive efforts to discover new therapeutic targets and new cancer drugs (11,12).

Glucose is the most important energy source for normal cells and tumors (13-15). Due to its polar hydrophilicity, glucose is not permeable to the hydrophobic plasma membrane and requires a specialized family of transmembrane transport proteins, the glucose transport proteins or GLUTs (GLUT1-14), to facilitate cellular entry (16) and cellular trapping as glucose 6-phosphate. Insulin-dependent overexpression of GLUT1 appears to be common in several cancers, including colorectal, lung, breast, ovarian, squamous cell, and glioblastoma cancers (17,18). In CRC cells, for example, the expression of GLUT1 was significantly increased, leading to increased uptake of glucose. GLUT1 inhibitors, including BAY-876, STF-31, WZB-117, exhibit promising anticancer activities in multiple cancer cells (19-21) by blocking glucose uptake and hence glycolysis crucial for cancer cell growth.

BAY-876 (i.e., N⁴-[1-(4-cyanobenzyl)-5-methyl-3-(trifluoromethyl)-1H-pyrazol-4-yl]-7-fluoroquinoline-2,4-dicarboxamide), is the first, highly selective GLUT1 inhibitor that reduced glucose uptake and inhibited tumor growth (22). BAY-876 is unique in its single-digit, nanomolar potency and selectivity for the GLUT1 transporter (i.e., by a factor >100 for GLUT1 in comparison with GLUT2, GLUT3 and GLUT4). It is also orally available when adminstered in vivo. In a SKOV-3 ovarian cancer xenograft mouse model, BAY-876 treatment at a dose of 4.5 mg/kg/day for 4 weeks reduced the average tumor volumes and tumor weights by 68% and 66%, respectively, and also was effective in inhibiting cancer growth in vivo. However, BAY-876 treatment resulted in an average 18% weight loss compared to the control group, indicating that BAY-876 is somewhat toxic to mice at this dose (23) (i.e., it has some adverse effects on normal cells). We anticipated that one means for increasing the therapeutic window for GLUT1 inhibitors would be to use them in a combination therapy that would cause the blockade of the GLUT1 transporter and inhibition of ETC.

For cancer cells, glycolysis alone could not provide enough energy to sustain cell growth. In addition to glucose, cancer growth also relies on other energy sources, including glutamine and fatty acids. As an example, inhibition of a key glutamine metabolism enzyme, glutamase, with CB-839, had synergistic effects with a GLUT1-3 inhibitor, Glutor, on CRC cells (24). The glucose, glutamine and fatty acid metabolic products are processed through the citric acid (TCA or Krebs) cycle and oxidative phosphoryaltion (OXPHOS) in the mitochondria (FIG. 1A). Recent studies indicated that a majority of tumor cells, particularly cancer stem cells (CSCs), depended on OXPHOS for ATP production (25-27), a finding that indicated that OXPHOS contributes to cancer development (28).

The OXPHOS metabolic pathway generates ATP by transporting electrons to a series of transmembrane protein complexes in the inner mitochondrial membrane, also known as the electron transport chain (ETC), in which NADH, FADH2 and succinate act as electron donors and in which protons are pumped from the mitochondrial matrix into the intermembrane space by Complexes I, II, III and IV as electrons pass through multiprotein ETC complexes I through IV (29). Mitochondrial Complex I is the first committed step in the mitochondrial electron transport chain from NADH and the gatekeeper between glycolysis/TCA substrate metabolism and the mitochondrial electron transport cascade leading to cellular ATP production (30).

Although prior investigators developed Complex I inhibitors, many of these inhibitors are either too toxic or too inefficient in blocking OXPHOS. Rotenone, for example, a lipophilic natural compound that was once widely used as an insecticide and fish killer, is a potent inhibitor of mitochondrial respiratory chain (MRC) complex I. Its mechanism of action includes inhibition of electron transfer from the iron-sulfur center in Complex I to ubiquinone, leading to blocked oxidative phosphorylation and restricted synthesis of ATP. Its toxicity, however, led to its withdrawal from the market in many countries (31,32). Recently, metformin, a first-line medication for the treatment of type II diabetes, particularly for overweight patients, reduced the risk of cancer (33-36). Metformin also targeted Complex I and reduced ATP production in the mitochondria, outcomes resulting activation of AMPK that monitors energy homeostasis by regulating downstream cellular events, such as mTOR signaling and lipid and glucose metabolism (37). However, the potency of metformin is very low and it has many other potential targets.

Accordingly, there remains a need in the art for improved energy metabolism inhibitors for cancer monotherapy or combination therapy with GLUT1 inhibitors that possess selectivity for cancer cells over normal cells.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes compounds and methods for use in the treatment of cancer.

Provided herein are compounds for inhibiting mitochondrial complex I. In some embodiments, the compound includes an isoflavonoid. In some embodiments, the compound includes a structure according to Formula I, and analogs and salts thereof:

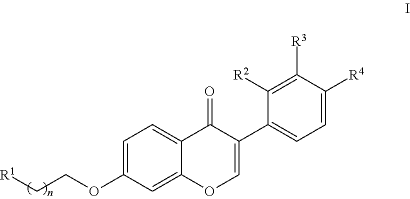

where R¹ includes a heterocycloalkyl or a secondary aminoalkyl; each of R², R³, and R⁴ independently includes H, halogen, or alkoxy; and n is 1, 2, 3, 4, 5, 6, 7, 8, 9, or 10; or any combination, sub-combination, range, or sub-range thereof.

In some embodiments, the compounds disclosed herein inhibit mitochondrial complex I. Complex I functions as part of the electron transport chain that leads ultimately to the production of adenosine triphosphate (ATP), the “energy currency” of cells. Inhibiting complex I deprives rapidly growing cancer cells of the energy supply needed for growth. Thus, in some embodiments, the compounds disclosed herein inhibit the rapid proliferation of multiple varieties of cancer cells. Accordingly, also provided herein, are methods of treating cancer in a subject, the methods including administering a pharmaceutically effective amount of one or more of the compounds disclosed herein to a subject in need thereof. Suitable cancers include, but are not limited to, colon cancer, liver cancer, ovarian cancer, lung cancer, prostate cancer, and breast cancer.

In some embodiments, the compounds disclosed herein exhibit synergistic effects with other cancer energy metabolism inhibitors. Other cancer energy metabolism inhibitors include, but are not limited to, the GLUT1 inhibitor and BAY-876.

Additionally or alternatively, in some embodiments, the compounds disclosed herein may be administered for treatment of other conditions, such as, but not limited to, in immunotherapy, treatment for diabetes, and obesity.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIGS. 1A-C show graphs and images illustrating that GLUT1 is a therapeutic target for CRC. (A) Expression of Glucose transporters in CRC. Only GLUT1 was overexpression in CRC. Red: Tumor. Grey: Normal tissue. (B) Structure of GLUT1 inhibitor BAY-876. (C) BAY-876 inhibited proliferation of CRC cell line LS174T and patient-derived CRC cell line Pt2377.

FIGS. 2A-F show graphs and images illustrating identification of a semi-synthetic isoflavone that enhances the efficacy of BAY-876 on CRC cell inhibition. (A) Diagram of cell energy metabolism pathways. (B) Structure of DBI-1. (C-D) DBI-1 inhibited CRC cell proliferation. (E-F) Combinational effects of DBI-1 and BAY-876 on CRC cell proliferation.

FIGS. 3A-B show graphs illustrating synergistic effects of DBI-1 and BAY-876 on CRC cells. (A-B) DBI-1 and BAY-876 synergistically inhibited CRC cell proliferation. The Bliss synergy scores were 21.809 for LS174T cells and 19.619 for Pt2377 cells.

FIGS. 4A-B show images illustrating the effects of DBI-1 and BAY-876 on cell signaling pathways in CRC cells. (A) DBI-1 (3 μM) inhibit Wnt signaling and activated AMPK signaling in CRC cells. (B) Combinational effects on DBI-1 (1 μM) and BAY-876 (0.1 mM) on Wnt and AMPK signaling in CRC cells.

FIGS. 5A-D show graphs illustrating that DBI-1 is a novel mitochondrial complex I inhibitor. (A) Standard Seahorse assay. (B) Replacing oligomycin with DBI-1 decreased OCR but the decreased OCR could not be rescued by the mitochondrial uncoupler, FCCP, suggesting that DBI-1 is not an ATP synthase inhibitor. (C) Replacing Rotenone/Antimycin A with DBI-1 decreased OCR, suggesting that DBI-1 inhibited mitochondrial complex I or complex III. (D) OCR inhibited by Rotenone or DBI-1 could be rescued by succinate, the substrate for complex II (succinate dehydrogenase or succinate-coenzyme Q reductase), substrate, suggesting that DBI-1 is a complex I inhibitor.

FIGS. 6A-B show graphs illustrating combinational effects of BAY-876 with other energy metabolism inhibitors on CRC cells. (A-B) Combinational effects of BAY-876 with Rotenone and metformin, two of the well-known complex I inhibitors.

FIGS. 7A-B show images and a graph illustrating that DBI-1 inhibited mouse CRC organoids. (A) Mouse CRC organoids treated with DMSO or DBI-1. (B) DBI-1 significantly inhibited the growth of CRC organoids.

FIGS. 8A-B show images and a graph illustrating combinational effects of DBI-1 and BAY-876 on mouse CRC organoid formation. (A) Formation of Mouse CRC organoids. (B) Combinational effects of DBI-1 and BAY-876 on mouse CRC organoid formation.

FIGS. 9A-E show graphs and images illustrating the in vivo effects of DBI-1 and BAY-876 on CRC cell xenografts in SCID mice. (A-B) DBI-1 and BAY-876 synergistically inhibited LS174T xenografts growth in SCID mice, without gross toxic effects in terms of body weight. (C) Tumors after two week treatment. (D-E) H&E and Ki67 staining of tumor sections.

FIG. 10 shows a graph illustrating the effects of certain DBI-1 analogs (DBI-1a: 3-(4-Bromophenyl)-7-(4-((2-(dimethylamino)ethyl)(methyl)amino)butoxy)-4H-chromen-4-one bishydrochloride [DBI-1-(HCl)₂]; DBI-1b: 3-(4-Chlorophenyl)-7-(4-((2-(dimethylamino)ethyl)(methyl)amino)butoxy)-4H-chromen-4-one bishydrochloride [DBI-1 (HCl)₂]; DBI-1c: 3-(4-Fluorophenyl)-7-(4-((2-(dimethylamino)ethyl)(methyl)amino)butoxy)-4H-chromen-4-one bishydrochloride [DBI-1·(HCl)₂]) on LS174T CRC cell proliferation.

FIGS. 11A-FIG. 11. SAR study of DBI-1 analogs. A. Synthetic method. Reagents and conditions: (a) Resorcinol, DMF, PCl5, BF3·Et2O, 85° C., 4 h; (b) Dibromo alkane, K2CO3, DMF, 80° C., 3 h; (c) Amine, DIPEA, NaI, DMF, 60° C., 4 h. B. DBI-1 and its analogues activated AMPK phosphorylation in LS174T cells. C. DBI-1 and DBI-6 (3 μmol/L) increased the phosphorylation of AMPK (p-AMPK) and ACC (p-ACC), markers for AMPK activation, and reduced the phosphorylation of P70S6K (p-P70S6K) and S6 (p-S6), markers for the mTOR signaling, and reduced the expression of Axin2 and c-Myc, key markers for the Wnt signaling, in LS174T CRC cells after 24 hours treatment.

FIGS. 12A-12D. Identification of DBI-6 as a novel inhibitor of mitochondrial complex 1. A, Standard Seahorse OCR assay. Oligomycin inhibited ATP-linked respiration and decreased OCR. FCCP rescued oligomycin-inhibited OCR and the FCCP-induced OCR was inhibited by rotenone and antimycin A. B, Oligomycin was replaced with DBI-6, OCR was inhibited but the decreased OCR could not be rescued by the addition of FCCP, indicating that DBI-6 is not a mitochondrial uncoupler or a complex V/ATP synthase inhibitor. C, Replacing rotenone/antimycin A with DBI-6 decreased OCR, indicating that DBI-6 inhibited mitochondrial complex I or complex III. D, Succinate, the substrate for complex II (succinate dehydrogenase or succinate-coenzyme Q reductase), could rescue the inhibited OCR caused by rotenone or DBI-6, indicating that DBI-6 is a complex I inhibitor.

FIGS. 13A-13F. DBI-6 and BAY-876 synergically inhibited CRC cells in vitro. A and B, DBI-6 inhibited colorectal cancer cell LS174T and HCT116 proliferation. The IC50 of DBI-6 to inhibit the proliferation of LS174T cells and HCT116 cells were 1.14 μmol/L and 0.53 μmol/L, respectively. C and D, Glut1 inhibitor, BAY-876, inhibited colorectal cancer cell LS174T and HCT116 proliferation. The IC50 of BAY-876 to inhibit the proliferation of LS174T cells and HCT116 cells were 0.13 μmol/L and 0.17 μmol/L, respectively. E and F, Combinatorial effects of DBI-6 and BAY-876 on CRC cell proliferation. The cells were treated with DBI-6 or BAY-876 at indicated concentrations for 5 days, and viable cells were counted.

FIGS. 14A-14B. Synergistic effects of DBI-6 and BAY-876 on CRC cells. A and B, DBI-6 and BAY-876 synergistically inhibited the proliferation of colorectal cancer cell. The cells were treated with DBI-6 and BAY-876 at indicated concentrations for 5 days, and viable cells were counted. Synergy scores were measured using SynergyFinder with Bliss as a reference model. The Bliss synergy scores for drug combinations were 25.414 for LS174T cells and 14.966 for HCT116 cells.

FIGS. 15A-15D. Synergistic effects of DBI-6 and BAY-876 on apoptosis of LS174T cell by flow cytometry for Annexin-V-FITC and propidium iodide (PI) dual labeling. A, Cells were treated with DMSO. The apoptosis rate was 17.28%. B, Cells were treated with DBI-6 at 3 μmol/L. The percentage of apoptotic cells was 30.61%. C, Cells were treated with BAY-876 at 1 μmol/L. The apoptosis rate was 34.57%. D, Cells were treated with DBI-6 (3 μmol/L) and BAY-876 (1 μmol/L). The combination of DBI-6 with BAY-876 resulted in a significant increase in apoptosis rate of 74.5%.

FIG. 16. Effects of DBI-6 and BAY-876 on cell signaling pathways in colorectal cancer cells. Combination of DBI-6 (3 μmol/L) and GLUT1 inhibitor BAY-876 (1 μmol/L) resulted in enhanced depletion of Axin2 and c-Myc, increase of p-AMPK and p-ACC, and depletion of p-P70S6K and p-S6.

FIGS. 17A-17C. Differential expression and pathway analyses. A, Volcano plot of differential expression analysis of LS174T cell with genes significantly up- and downregulated marked in red and blue, respectively. The X-axis represents the fold change of the difference after conversion to log 2, and the Y-axis represents the significance value after conversion to-log 10. Qvalue ≤0.05 and |log 2FC|≥1. B, Histogram of Go enrichment classification. The X-axis represents the number of genes annotated to the GO entry, and the Y-axis represents the GO functional classification. C, The KEGG pathway classification analysis of differentially expressed genes. The X-axis is the number of genes annotated to a category of KEGG Pathway, and the Y-axis is the category of KEGG Pathway.

FIGS. 18A-18F. DBI-6 and the GLUT1 inhibitor BAY-876 synergistically inhibited colon cancer cell xenografts in vivo. A, B and C, The combination of intraperitoneal DBI-6 at 40 mg/kg/day and oral BAY-876 at 3 mg/kg/day and the combination of administration of DBI-6 at 40 mg/kg/day together with ketogenic diet feeding significantly inhibited tumor growth in Rag1 mice, without gross toxic effects in terms of body weight. D, Tumors after 12-day treatment. E and F, H&E and Ki67 staining of tumor sections (a: control; b: BAY-876; c: Keto diet; d: DBI-6; e: DBI-6+BAY-876; f: DBI-6+Keto diet).

FIGS. 19A-19. Combination of DBI-6 with BAY-876 reduced blood glucose, plasma TG, T-CHO and LDL-C. A, Blood glucose tested from Rag1 mice tails. B, C, D and E, The plasma TG, T-CHO, LDL-C and HDL-C tested in tumor bearing animals. Blood obtained via retro orbital eye bleeds prior to sacrifice. Blood samples spun and plasma separated.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes compounds and methods for use in the treatment of cancer.

Provided herein are compounds for inhibiting mitochondrial complex I. In some embodiments, the compound includes an isoflavonoid. In some embodiments, the compound includes a structure according to Formula I, and analogs and salts thereof:

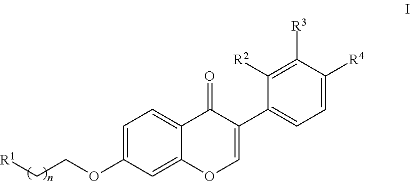

where R¹ includes a heterocycloalkyl or a secondary aminoalkyl; each of R², R³, and R⁴ independently includes H, halogen, or alkoxy; and n is 1, 2, 3, 4, 5, 6, 7, 8, 9, or 10; or any combination, sub-combination, range, or sub-range thereof.

In some embodiments of the compound, R¹ is a heterocycloalkyl including a five or six membered ring and may be substituted or unsubstituted. In some embodiments, the heterocycloalkyl includes one heteroatom. In some embodiments, the heterocycloalkyl includes two or more heteroatoms.

In some embodiments, the six membered heterocycloalkyl includes a structure according to Formula II.

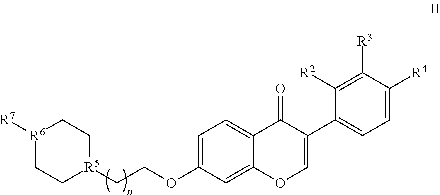

where each of R⁵ and R⁶ independently include N, S, or O; and R⁷ includes H or hydroxyalkyl.

Examples of compounds including a six-membered heterocycloalkyl provided in accordance with the presently-disclosed subject matter include, but are not limited to, those set forth in Table 1.

TABLE 1
Examples of Compounds including Six-Membered Heterocycloalkyl

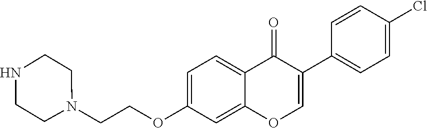 [SMILES: O=c1c(-c2ccc(Cl)cc2)coc2cc(OCCN3CCNCC3)ccc12]

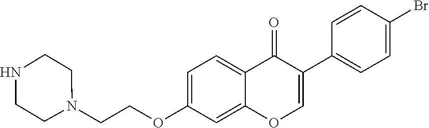 [SMILES: O=c1c(-c2ccc(Br)cc2)coc2cc(OCCN3CCNCC3)ccc12]

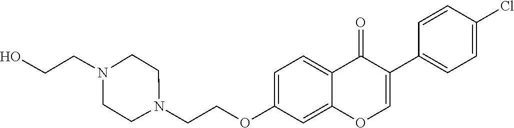 [SMILES: O=c1c(-c2ccc(Cl)cc2)coc2cc(OCCN3CCN(CCO)CC3)ccc12]

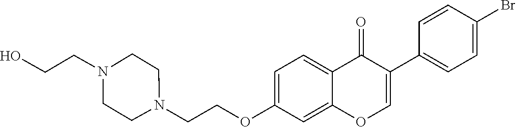 [SMILES: O=c1c(-c2ccc(Br)cc2)coc2cc(OCCN3CCN(CCO)CC3)ccc12]

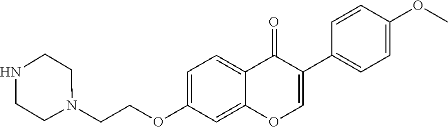 [SMILES: COc1ccc(-c2coc3cc(OCCN4CCNCC4)ccc3c2=O)cc1]

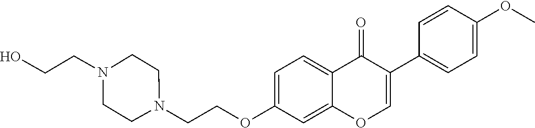 [SMILES: COc1ccc(-c2coc3cc(OCCN4CCN(CCO)CC4)ccc3c2=O)cc1]

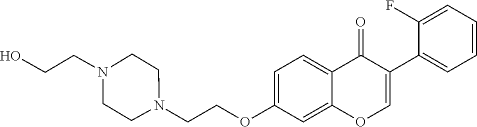 [SMILES: O=c1c(-c2ccccc2F)coc2cc(OCCN3CCN(CCO)CC3)ccc12]

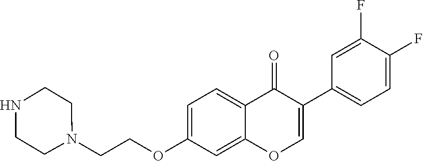 [SMILES: O=c1c(-c2ccc(F)c(F)c2)coc2cc(OCCN3CCNCC3)ccc12]

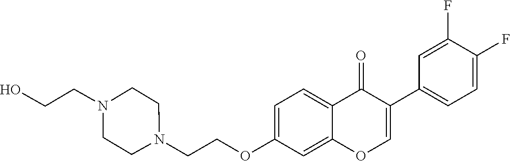 [SMILES: O=c1c(-c2ccc(F)c(F)c2)coc2cc(OCCN3CCN(CCO)CC3)ccc12]

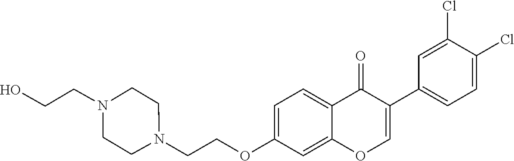 [SMILES: O=c1c(-c2ccc(Cl)c(Cl)c2)coc2cc(OCCN3CCN(CCO)CC3)ccc12]

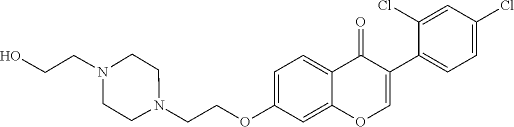 [SMILES: O=c1c(-c2ccc(Cl)cc2Cl)coc2cc(OCCN3CCN(CCO)CC3)ccc12]

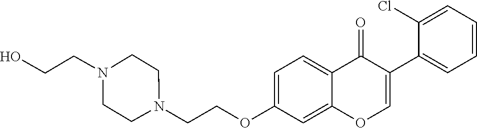 [SMILES: O=c1c(-c2ccccc2Cl)coc2cc(OCCN3CCN(CCO)CC3)ccc12]

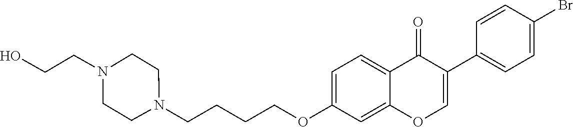 [SMILES: O=c1c(-c2ccc(Br)cc2)coc2cc(OCCCCN3CCN(CCO)CC3)ccc12]

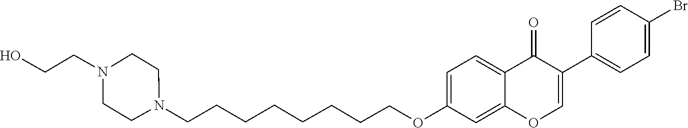 [SMILES: O=c1c(-c2ccc(Br)cc2)coc2cc(OCCCCCCCCN3CCN(CCO)CC3)ccc12]

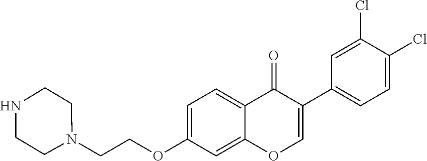 [SMILES: O=c1c(-c2ccc(Cl)c(Cl)c2)coc2cc(OCCN3CCNCC3)ccc12]

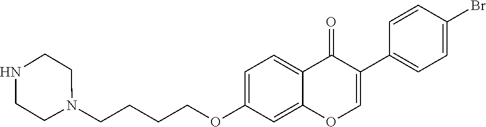 [SMILES: O=c1c(-c2ccc(Br)cc2)coc2cc(OCCCCN3CCNCC3)ccc12]

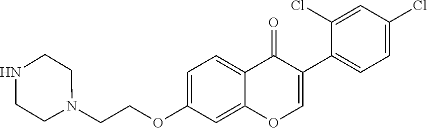 [SMILES: O=c1c(-c2ccc(Cl)cc2Cl)coc2cc(OCCN3CCNCC3)ccc12]

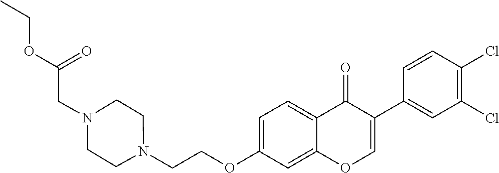 [SMILES: CCOC(=O)CN1CCN(CCOc2ccc3c(=O)c(-c4ccc(Cl)c(Cl)c4)coc3c2)CC1]

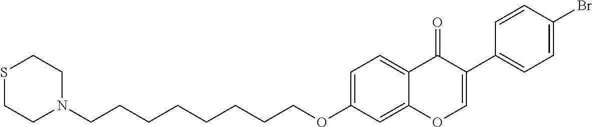 [SMILES: O=c1c(-c2ccc(Br)cc2)coc2cc(OCCCCCCCCN3CCSCC3)ccc12]

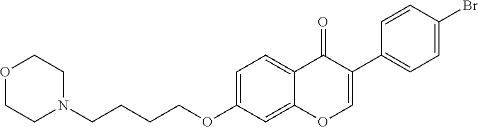 [SMILES: O=c1c(-c2ccc(Br)cc2)coc2cc(OCCCCN3CCOCC3)ccc12]

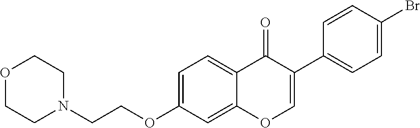 [SMILES: O=c1c(-c2ccc(Br)cc2)coc2cc(OCCN3CCOCC3)ccc12]

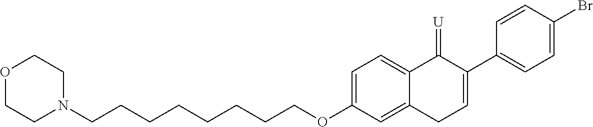

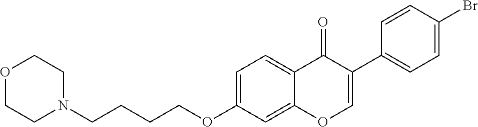 [SMILES: O=c1c(-c2ccc(Br)cc2)coc2cc(OCCCCN3CCOCC3)ccc12]

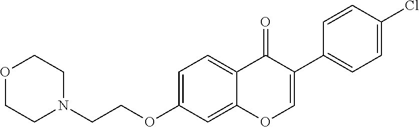 [SMILES: O=c1c(-c2ccc(Cl)cc2)coc2cc(OCCN3CCOCC3)ccc12]

In some embodiments, the five membered heterocycloalkyl includes a structure according to Formula III:

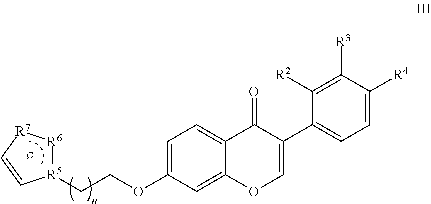

where each of R⁵, R⁶, and R⁷ independently include C or N. In some embodiments, two of R⁵, R⁶, and R⁷ include N and the third includes C. Examples of compounds including a five-membered heterocycloalkyl provided in accordance with the presently-disclosed subject matter include, but are not limited to, those set forth in Table 2.

TABLE 2
Examples of Compounds including Five-Membered Heterocycloalkyl

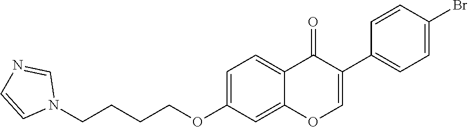 [SMILES: O=c1c(-c2ccc(Br)cc2)coc2cc(OCCCCn3ccnc3)ccc12]

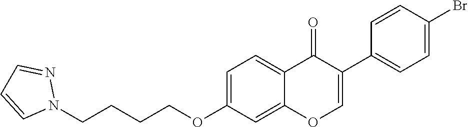 [SMILES: O=c1c(-c2ccc(Br)cc2)coc2cc(OCCCCn3cccn3)ccc12]

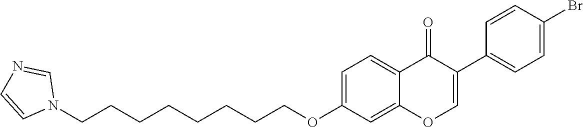 [SMILES: O=c1c(-c2ccc(Br)cc2)coc2cc(OCCCCCCCCn3ccnc3)ccc12]

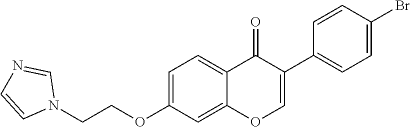 [SMILES: O=c1c(-c2ccc(Br)cc2)coc2cc(OCCn3ccnc3)ccc12]

In some embodiments of the compound, R¹ is a secondary aminoalkyl, which is inclusive of, but not limited to, linear secondary amino alkyls. As will be appreciated by the skilled artisan, a secondary amino alkyl does not include any primary amines, such as any primary aliphatic amines. As is well-established in the art, a primary amine has one carbon bound to the nitrogen, which a secondary amine has two carbons bound to the nitrogen.

In some embodiments, the secondary aminoalkyl includes a structure according to Formula IV:

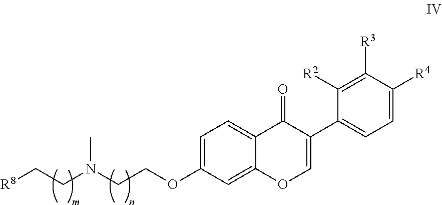

wherein m is from 1 to 10, and wherein R⁸ is OH, N(alkyl)₂, aromatic ring, piperidine or analogs thereof, morpholine, or analogs thereof, or piperazine or analogs thereof.

Examples of compounds including a linear secondary aminoalkyl provided in accordance with the presently-disclosed subject matter include, but are not limited to, those set forth in Table 3.

TABLE 3
Examples of Compounds including Linear Secondary Amino Alkyl

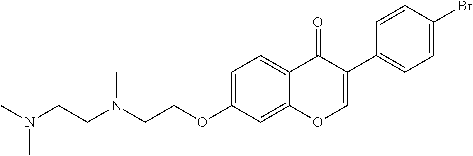 [SMILES: CN(C)CCN(C)CCOc1ccc2c(=O)c(-c3ccc(Br)cc3)coc2c1]

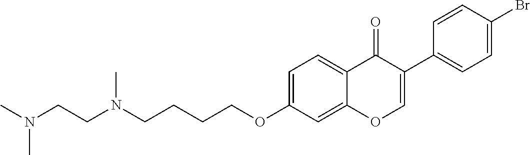 [SMILES: CN(C)CCN(C)CCCCOc1ccc2c(=O)c(-c3ccc(Br)cc3)coc2c1]

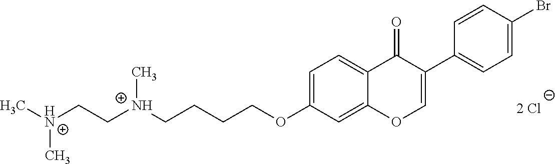

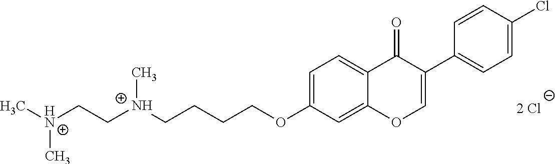

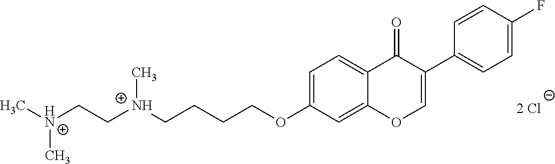

In some embodiments, the compounds disclosed herein inhibit mitochondrial complex I. Complex I functions as part of the electron transport chain that leads ultimately to the production of adenosine triphosphate (ATP), the “energy currency” of cells. Inhibiting complex I deprives rapidly growing cancer cells of the energy supply needed for growth. Thus, in some embodiments, the compounds disclosed herein inhibit the rapid proliferation of multiple varieties of cancer cells. Accordingly, also provided herein, are methods of treating cancer in a subject, the methods including administering a pharmaceutically effective amount of one or more of the compounds disclosed herein to a subject in need thereof. Suitable cancers include, but are not limited to, colon cancer, liver cancer, ovarian cancer, lung cancer, prostate cancer, and breast cancer.

In some embodiments, the compounds disclosed herein exhibit synergistic effects with other cancer energy metabolism inhibitors. Other cancer energy metabolism inhibitors include, but are not limited to, the GLUT1 inhibitor and BAY-876.

Additionally or alternatively, in some embodiments, the compounds disclosed herein may be administered for treatment of other conditions, such as, but not limited to, in immunotherapy, treatment for diabetes, and obesity.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

Although any methods, devices, and materials similar or equivalent to those described herein can be used in the practice or testing of the presently-disclosed subject matter, representative methods, devices, and materials are described herein.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, nomenclature for compounds, including organic compounds, can be given using common names, IUPAC, IUBMB, or CAS recommendations for nomenclature.

When one or more stereochemical features are present, Cahn-Ingold-Prelog rules for stereochemistry can be employed to designate stereochemical priority, ElZ specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration.

Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, the terms “analog” and “derivative” refer to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

The terms “alkoxy” and “alkoxyl” as used herein to refer to an alkyl or cycloalkyl group bonded through an ether linkage; that is, an “alkoxy” group can be defined as —OR¹ where R¹ is alkyl or cycloalkyl as defined above. “Alkoxy” also includes polymers of alkoxy groups as just described; that is, an alkoxy can be a polyether such as —OR¹—OR² or —OR¹—(OR²)ₐ—OR³, where “a” is an integer of from 1 to 200 and R¹, R², and R³ are alkyl and/or cycloalkyl groups.

The term “alkyl” as used herein is a branched or unbranched saturated hydrocarbon group, such as methyl, ethyl, propyl, isopropyl, butyl, isobutyl, s-butyl, i-butyl, pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group is acyclic. The alkyl group can be branched or unbranched. Unless explicitly stated otherwise, the alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “alkoxyalkyl” specifically refers to an alkyl group that is substituted with one or more alkoxy groups (e.g., —CH₂OCH₃), as described below. The term “arylalkyl” specifically refers to an alkyl group that is substituted with one or more aryl groups (e.g., —CH₂C₆H₅), as described below. When “alkyl” is used in one instance and a specific term such as “hydroxyalkyl” is used in another, it is not meant to imply that the term “alkyl” does not also refer to specific terms such as “hydroxyalkyl” and the like.

A “primary amine” is an amine having one carbon bound to the nitrogen. A “secondary amine” is an amine having two carbons bound to the nitrogen.

The term “aromatic group” as used herein refers to a ring structure having cyclic clouds of delocalized π electrons above and below the plane of the molecule, where the π clouds contain (4n±2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

The terms “halo,” “halide,” or “halogen” as used herein refer to fluoro, chloro, bromo, and iodo groups.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridine, pyridazine, pyrimidine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like.

The term “hydroxyl” as used herein is represented by the formula —OH.

The presently-disclosed subject matter is further illustrated by the following specific but non-limiting examples. The following examples may include compilations of data that are representative of data gathered at various times during the course of development and experimentation related to the present invention.

### EXAMPLES

### Example 1

### Materials and Methods

General Chemistry Methods.—Chemicals and solvents were obtained from Sigma Aldrich (St. Louis, MO, USA) with the exception of resorcinol and 4-bromophenylacetic acid (Acros Organic, Carlsbad, CA, USA), 1,4-dibromobutane (Oakwood Chemical, Estill, SC, USA), sodium iodide (Fisher Scientific, Waltham, MA, USA), and N,N,N′-trimethyl-1,2-ethylenediamine (Toyko Chemical, Tokyo, Japan). Nuclear magnetic resonance spectra were determined in DMSO-d6 using Varian instruments (¹H, 400; ¹³C, 100 Mz; Varian, Inc., Palo Alto, CA, USA). High resolution electrospray ionization (ESI) mass spectra were recorded on a LTQ-Orbitrap Velos mass spectrometer (Thermo Fisher Scientific, Waltham, MA, USA). The FT resolution was set at 100,000 (at 400 m/z). Samples were introduced through direct infusion using a syringe pump with a flow rate of 5 μL/min. Purity was established by combustion analyses performed by AμLantic Microlabs, Inc. (Norcross, GA, USA).

3-(4-Bromophenyl)-7-hydroxy-4H-chromen-4-one (43).—To a mixture of 1.1 g (10 mmol, 1 eq) of resorcinol 2.37 g and (11 mmol, 1.1 eq) of 4-bromophenylacetic acid was added 10 mL (11.4 g, 80 mmol) of boron tribromide etherate. The mixture was heated at 85° C. for 4 h under a nitrogen atmosphere to afford a wine-red solution. The solution was cooled to 0° C., and 10 mL of anhydrous N,N-dimethylformamide (DMF) was added dropwise. To 20 mL of anhydrous DMF in a separate flask at 0° C. was added 3.12 g (15 mmol, 1.5 eq) of phosphorous pentachloride in portions over a 10 min period to afford an orange solution. This orange solution was stirred sequentially for 30 min at 25° C., 30 min at 55° C., and 30 min at 25° C. to afford a solid suspended in an orange solution. To this orange mixture was added the wine-red colored solution at 25° C. under a nitrogen atmosphere. The solution was stirred for 3 h at 25° C. and poured into 80 mL of boiling 0.1M aqueous hydrochloric acid. The resulting precipitate was collected in a Buchner funnel and washed with water. The crude product was dried under vacuum and recrystallized from absolute ethanol to afford 801 mg (25%) of 3-(4-bromophenyl)-7-hydroxy-4H-chromen-4-one as a peach-colored solid with NMR data consistent with literature values (43).

7-(4-Bromobutoxy)-3-(4-bromophenyl)-4H-chromen-4-one.—The procedure of Gao, et al. (44), was repeated using a mixture of 100 mg (0.32 mmol, 1 eq) of 3-(4-bromophenyl)-7-hydroxy-4H-chromen-4-one, 341 mg (1.58 mmol, 5 eq) of 1,4-dibromobutane and 109 mg (0.79 mmol, 2.5 eq) of anhydrous potassium carbonate in 1.6 mL of anhydrous DMF at 25° C. under a nitrogen atmosphere. The cloudy orange-yellow solution was heated for 1.5 h at 80° C. The reaction was quenched with 50 mL of cold water to afford a white precipitate. The precipitate was collected in a Buchner funnel and washed successively with water and ether to afford after drying in vacuo 75 mg (53%) of 7-(4-bromobutoxy)-3-(4-bromophenyl)-4H-chromen-4-one that was sufficienμLy pure to be used direcμLy in the next reaction: ¹H NMR (400 MHz, CDCl3) δ 1.98-2.07 (s, 4H, BrCH₂CH₂CH₂CH₂O), 3.49 (t, 2H, BrCH₂CH₂CH₂CH₂O), 4.09 (t, 2H, BrCH₂CH₂CH₂CH₂O), 6.83 (d, J=2.4 Hz, 1H, H-6), 6.98 (dd, J=2.4 Hz and 9.2 Hz, 1H, H-8), 7.41-7.46 (m, 2H, H-2′), 7.52-7.57 (m 2H, H-3′), 7.93 (s, 1H, H-2), 8.18 (d, J=8.8 Hz, H-5). HRMS (ESI) Calcd for C₁₉H₁₇O₃⁷⁹Br₂ [MH+]; C₁₉H₁₇O₃⁷⁹Br⁸¹Br [MH+]; and C₁₉H₁₇O₃⁸¹Br₂ [MH+] in a 1:2:1 ratio: 450.9539; 452.9518; and 454.9498, respectively. Found: 450.9545; 452.9524; and 454.9503, respectively.

3-(4-Bromophenyl)-7-(4-((2-(dimethylamino)ethyl)(methyl)amino)butoxy)-4H-chromen-4-one (DBI-1).—To a mixture of 391 mg (0.86 mmol, 1 eq) of 7-(4-bromobutoxy)-3-(4-bromophenyl)-4H-chromen-4-one, 129 mg (0.86 mmol, 1 eq) of sodium iodide and 0.5 mL (3.5 eq) of N,N-diisopropylethylamine in 2.2 mL of anhydrous DMF under a nitrogen atmosphere was added 132 mg (1.3 mmol, 1.5 eq) of N,N,N′-trimethyl-1,2-ethylenediamine in one portion. The mixture was stirred for 6 h at 60° C., cooled, and diluted with 120 mL of dichloromethane. The organic solution was washed successively with two 25 mL portions of water and 25 mL of saturated sodium chloride solution. The organic layer was dried over anhydrous magnesium sulfate, filtered and concentrated to afford 380 mg of crude product that was chromatographed on silica gel F254 preparative layer plates using 1:20:200 concentrated aqueous ammonium hydroxide solution-methanol-dichloromethane to afford (Rf=0.3) 252 mg (62%) of the “free base” of DBI-1 as a wax-like, sticky, pale-yellow solid: ¹H NMR (400 MHz, CDCl3) δ 1.6-1.9 (s, 4H, N(CH₃)CH₂CH₂CH₂CH₂O), 2.23 and 2.24 (two s, 9H, (CH₃)₂NCH₂CH₂N(CH₃)), 2.38-2.52 (m, 6H, (CH₃)₂NCH₂CH₂N(CH₃)CH₂), 4.07 (t, J=6.4 Hz, 2H, N(CH₃)CH₂CH₂CH₂CH₂O), 6.83 (d, J=2.4 Hz, 1H, H-6), 6.98 (dd, J=2.4 Hz and 9.2 Hz, 1H, H-8), 7.4-7.58 (m, 4H, H-2′ and H-3′), 7.92 (s, 1H, H-2), 8.17 (d, J=9.2 Hz, H-5); ¹³C NMR (100 MHz, CDCl3) δ 23.73, 26.92, 42.51, 45.86, 55.63, 57.46, 57.84, 68.52, 100.63, 115.06, 118.15, 122.29, 124.23, 127.7, 130.51, 130.91, 131.58, 152.44, 157.92, 163.63, and 175.27; HRMS (ESI) Calcd for C₂₄H₃O⁷⁹BrN₂O₃ [MH+] and C₂₄H₃₀⁸¹BrN₂O₃[MH] in a 1:1 ratio: 473.1440 and 475.1419, respectively. Found: 473.1436 and 475.1420, respectively.

### 7-Hydroxy-3-phenyl-4H-chromen-4-one (1)

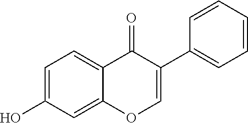 [SMILES: O=c1c(-c2ccccc2)coc2cc(O)ccc12]

To a mixture of catechol (10 mmol) and phenylacetic acid (11 mmol) was added boron trifluoride diethyl ether (80 mmol). The mixture was heated at 85° C. for 4 h. The reddish solution was cooled 10° C., and 10 mL of DMF was slowly added. To this mixture was added a second mixture produced from adding 20 mL of DMF to PCl5 (15 mmol) initially at 10° C. and then at 25° C. for 30 min, and finally at 55° C. for 30 min. These combined solutions were stirred at 25° C. for 3 h. The mixture was poured into 80 mL of boiling 0.1 M aqueous hydrochloric acid and the resulting precipitate was collected, washed with 50 mL of water, and recrystallized from anhydrous ethanol.

### 7-(2-Bromoethoxy)-3-phenyl-4H-chromen-4-one (2)

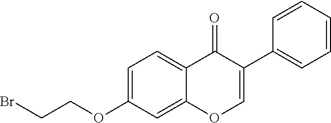 [SMILES: O=c1c(-c2ccccc2)coc2cc(OCCBr)ccc12]

To a solution of 2 mmol of 7-hydroxyisoflavonoid 1 in DMF (10 mL) was added anhydrous K₂CO₃ (5 mmol) and 1,2-dibromoethane (10.4 mmol) under a nitrogen atmosphere. The mixture was stirred for 3 h at 80° C. The mixture was cooled and poured into cold water. The precipitate was collected and washed successively with water and cold diethyl ether to afford 2 that was purified by crystallization and/or chromatography on silica gel.

### 3-Phenyl-7-(2-(piperazin-1-yl)ethoxy)-4H-chromen-4-one (3)

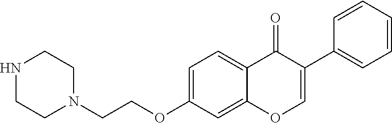 [SMILES: O=c1c(-c2ccccc2)coc2cc(OCCN3CCNCC3)ccc12]

To a solution of 2 (1 mmole) in DMF (10 mL) was added piperazine (2 mmol), NaI (1 mmol), and anhydrous K₂CO₃ (2 mmol) under a nitrogen atmosphere. The mixture was stirred for 2 h at 60° C. The mixture was cooled and poured into cold water (100 mL). The precipitate was collected and washed with cold water. The product was recrystallized from methanol to afford a white solid.

### 7-(2-(4-(2-hydroxyethyl)piperazin-1-yl)ethoxy)-3-phenyl-4H-chromen-4-one (4)

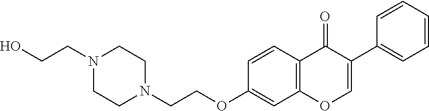 [SMILES: O=c1c(-c2ccccc2)coc2cc(OCCN3CCN(CCO)CC3)ccc12]

A mixture of 2 (1 mmol), 1-(2-hydroxyethyl)piperazine (1.2 mmol), NaI (1 mmol) and diisopropylethylamine (3.5 mmol) in DMF (10 mL) was stirred for 3 h at 60° C. under a nitrogen atmosphere. The mixture was cooled; concentrated, and purified by chromatography on silica gel using methanol-dichloromethane to afford 4.

### 3-(4-Bromophenyl)-7-(2-((2-(dimethylamino)(methyl)amino(ethoxy)-4H-chromen-4-one (DBI-2)

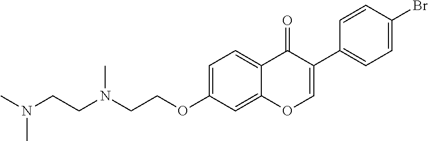 [SMILES: CN(C)CCN(C)CCOc1ccc2c(=O)c(-c3ccc(Br)cc3)coc2c1]

A mixture of 2 (1 mmol), N¹,N¹,N²-trimethylethane-1,2-diamine (1.2 mmol), NaI (1 mmol) and diisopropylethylamine (3.5 mmol) in DMF (10 mL) was stirred for 3 h at 60° C. under a nitrogen atmosphere. The mixture was cooled, concentrated chromatographed on silica gel using methanol-dichloromethane to afford DBI-2.

### 3-(4-Bromophenyl)-7-(2-((2-(dimethylamino)(methyl)amino(butoxy)-4H-chromen-4-one (DBI-1)

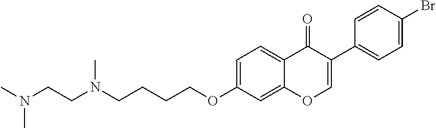 [SMILES: CN(C)CCN(C)CCCCOc1ccc2c(=O)c(-c3ccc(Br)cc3)coc2c1]

A mixture of 7-(4-bromobutoxy)-3-phenyl-4H-chromen-4-one (1 mmol), N¹,N¹,N²-trimethylethane-1,2-diamine (1.2 mmol), NaI (1 mmol) and diisopropylethylamine (3.5 mmol) in DMF (10 mL) was stirred for 3 h at 60° C. under a nitrogen atmosphere to afford, after column chromatography on silica gel using methanol-dichloromethane, DBI-1.

Biology Materials—Antibodies: Axin2 (#2151, Cell signaling technology), c-Myc (#1472-1, Epitomics), p-ACC (#11818, Cell signaling technology), ACC (#3676, Cell signaling technology), p-AMPK (#2535, Cell signaling technology), AMPK (#2532, Cell signaling technology), p-P70S6K (#9234, Cell signaling technology), P70S6K (#2708, Cell signaling technology), p-S6 (#4858, Cell signaling Cell signaling technology), S6 (#2317, Cell signaling technology), GAPDH (#GTX627408, GeneTex). Compounds: BAY-876 (N⁴-[1-(4-cyanobenzyl)-5-methyl-3-(trifluoromethyl)-1H-pyrazol-4-yl]-7-fluoroquinoline-2,4-dicarboxamide) was purchased from Medkoo Bioscience (#530485).

Cell lines and cell culture—LS174T cell line (LS174T-TR4) is a gift from Professor Hans Clevers and Marc van de Wetering. LS174T-TR4 cells were selected with resistance to blasticidin (45). Pt2377 is a primary colon cancer cell line established from a patient-derived xenograft (PDX) model (46,47). LS174T cells were cultured in RPMI1640 (Sigma, R8758) containing 5% Fetal Bovine Serum (Sigma F0926). Pt2377 cells were cultured in DMEM (Sigma, D6429) containing 10% Fetal Bovine Serum (Sigma F0926). Mycoplasma testing were performed using a sensitive PCR-based mycoplasma detection kit covering more than 200 species/strains of mycoplasmas (Biovision, K1476-100), and no mycoplasma contamination was found in the cell lines used in this work. All cells were cultured at 37° C. with 5% CO2 atmosphere in a water jacketed incubator (NuAire, Plymouth, MN).

Cell proliferation inhibition assay-Cell proliferation inhibition assay was performed as previously reported (48). All compounds to be tested were dissolved at 10 mM in DMSO. The cells were seeded into 12-well plates at a density of 40,000 cells per well in 1 mL of culture medium and were cultured overnight at 37° C. Either 1 μL of the compounds or the vehicle control (DMSO) was added to the cells. After 5 days, the medium was removed, and 200 μL of 0.25% trypsin was added. The cells were resuspended in 800 μL phosphate-buffered saline (PBS) and counted with Vi-CELL XR 2.03 (Beckman Coulter, Inc. USA). The ratio R of the number of viable cells in the compound-treated group to the number of viable cells in the DMSO-treated group was called relative growth rate, and the growth inhibition was calculated as (1−R)*100.

Calculation of synergy scores of BAY-876 and DBI-1—The cell proliferation inhibition assay described above was performed on LS174T and Pt2377 cells using 24-well plates to assess the inhibitory effect of BAY-876 with or without DBI-1 at predetermined concentrations. In LS174T cells, the final treatment concentrations of BAY-876 were 0 nM, 25 nM, 50 nM, 100 nM, 200 nM, and 400 nM in the presence or absence of DBI-1 at 0 nM, 250 nM, 500 nM, 1000 nM, 2000 nM, and 4000 nM. In Pt2377 cells, the final treatment concentrations for BAY-876 were 0 nM, 25 nM, 50 nM, 100 nM, 200 nM and 400 nM and for DBI-1 were 0 nM, 400 nM, 750 nM, 1500 nM and 3000 nM. Synergy scores were calculated using the default parameters of the SynergyFinder web application (version 2.0) (www.synergyfinder.org) using Bliss as the reference model (49).

Western blotting-Western blotting was carried out following previously reported procedures (50). Cells were incubated in 6-well plates and were treated with either DMSO or compounds dissolved in DMSO at indicated concentrations. Cells were lysed in 500 μl of lysis buffer: 50 mM HEPES, 100 mM NaCl, 2 mM EDTA, 1% (v/v) glycerol, 50 mM NaF, 1 mM Na3VO4, 1% (v/v) Triton X-100, with protease inhibitors (Sigma, P8340). The cell lysates were centrifuged (18,000×g, 10 min) and then 6× protein loading buffer was added to the suppernatants. The samples were boiled for 3 min and analyzed by standard Western blotting methods with the indicated antibodies.

Seahorse oxygen consumption rate assay-Seahorse assays were conducted in accordance with a published protocol (50). Mitochondrial bioenergetic measurements were performed using the Seahorse XFe96 Extracellular Flow Analyzer (Agilent Technologies, Santa Clara, CA, USA) to measure the oxygen consumption rate (OCR) in different respiratory states. 2.5×10⁴ LS174T cells in 80 μL medium were seeded in XF96 Cell Culture microplate and cultured overnight. Then cell culture media were replaced with Seahorse XF modified media before cells were treated. In the standard assay, cells were treated sequentially with 1 μM oligomycin, 1.0 μM FCCP, and a mixture of 1.0 μM rotenone and 1.0 μM antimycin A. To determine if compounds inhibited ETC complex V/ATP synthase, oligomycin was replaced with DMSO, or compounds dissolved in DMSO. To test whether compounds were ETC complex I or III inhibitors, rotenone and antimycin A were replaced with DMSO, or compounds in DMSO solution.

Mitochondrial ETC complex activity measurements using PMP-Measurements of mitochondrial ETC complex activity were performed using the Agilent Seahorse Assay with XF PMP (Agilent, Santa Clara, CA) according to the manufacturer's instructions. Mitochondrial assay solution (MAS) was prepared with 220 mM mannitol, 70 mM sucrose, 10 mM KH2PO4, 5 mM MgCl2, 2 mM HEPES, 1 mM EGTA, and 0.2% (w/v) fatty acid free BSA. Cells were gen Ly washed with MAS and cell media was replaced with MAS supplemented with 4 mM adenosine diphosphate (ADP), 10 mM pyruvate, and 1 nM PMP. After calibration, the test was prompμLy carried out by measuring baseline with two cycles of 0.5 min mixing, 0.5 min waiting, and 2 min measuring. Following the baseline measurements, DMSO, test compounds or 2 μM rotenone, 10 mM succinate, 2 μM antimycin A, and a combination of 10 mM ascorbate and 100 μM TMPD were sequentially injected. The experiment data were then normalized to cell counts using Biotek Cytation 1 (BioTek Instruments, Winooski, VT, USA).

Colon Cancer Organoids—The 3D tumor organoid model was established from Apcf/+/KrasLSL-G12D/Vil-Cre compound mutant mice (51). A 48-well drug screening was performed to assess how compounds affect the formation of organoid colony. Matrigel-containing organoids were digested by 300 uL dispase. Samples were centrifuged at 1000×g for 5 min to remove the Matrigel. The organoids in the supernatants were digested into single cells by 1 mL 0.25% Trypsin and washed with 10 mL of ADF12. Each well of a 48-well plate was pre-coated with 80 uL Matrigel, and 1000 cells in 60 uL Matrigel were added to each well. After the Matrigel solidified, 360 uL 3D complete medium (Advanced DMEM/F12 supplemented with 1×N-2, 1×B-27, 1 mmol/L N-acetylcysteine and 1% penicillin/streptomycin) was added to each well. Cells were treated with DMSO or the test compounds in DMSO for 7 days, and the total number of organoids larger than 50 μm in diameter were counted using a microscope and photographed. For the organoid growth inhibition assay, 1000 cells were seeded to each well as above. Either DMSO or the compounds in DMSO were added after three days when the organoids had formed. After three days, the media was replaced by fresh medium containing either DMSO or the compounds in DMSO, and cultured for another 3 days. The viability of the organoid was measured using the CellTiter-Glo® 3D Cell Viability Assay (Promega, G9681) according to the manufacturer's protocol.

In vivo evaluation of anti-cancer activity in LS174T xenografts—The mouse studies were conducted with the approval of the University of Kentucky Institutional Animal Care and Use Committee (2020-3531). All methods were carried out in compliance with the relevant guidelines and regulations according to protocols. The LS174T cells in exponential growth phase were trypsinized and resuspended in 50% Matrigel (Corning, Glendale, Arizona). Cells (1.2×10⁶) were subcutaneously injected on the lower flanks of severe combined immunodeficient mice. The tumor volumes were calculated based on the formula Length×width²/₂. When tumors reached an average volume of 100 mm³, the mice were divided into control and treatment groups (3 male and 3 female mice/group, two tumors on each mouse). After tumors were established, BAY-876 was given to the mice by gavage feeding to the BAY-876 group at the dose of 3 mg/kg/day. DBI-1 was daily intraperitoneally (i.p.) administered to the DBI-1 group at dose of 40 mg/kg mouse body weight. Mice in the combination treatment group were administered both oral BAY-876 at the dose of 3 mg/kg/day and i.p. DBI-1 at dose of 40 mg/kg mouse body weight. Tumors and mouse weights were measured every three days and the experiment was stopped at day 12. Tumors were isolated and fixed in 10% neutral buffered formalin. Tumor sections were analyzed with H&E and Ki-67 staining at Markey Cancer Center's Biospecimen Procurement and Translational Pathology Shared Resource Facility (BPTP SRF).

Statistics. Major biological assays were performed at least twice. Cell-based assays were performed in triplicates. Gene expression in TCGA database was analyzed using the GEPIA program (52). Statistical analysis was performed with GraphPad Prism 5. Data were presented as mean±SEM. Statistical significance among groups was calculated using the Student's two-tail t-test, and P<0.05 was considered statistically significant. Statistical significance was represented by * if P<0.05 and ** if P<0.01.

### Results

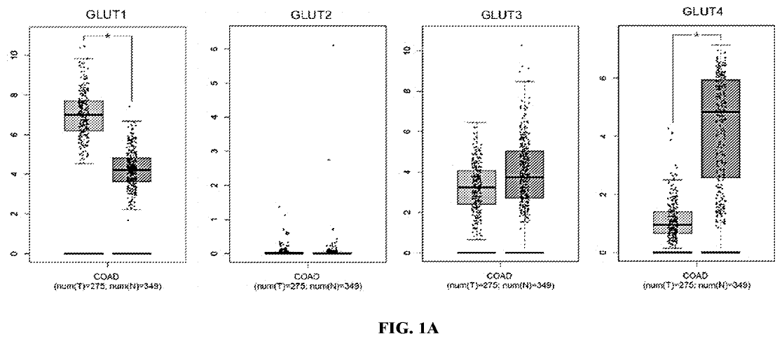

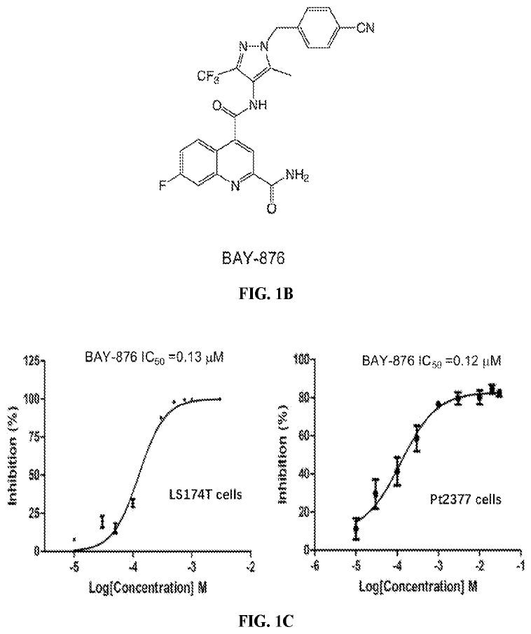

GLUT1 is a therapeutic target for CRC. There are four major types of glucose transporters, GLUT1-4. The expression of these genes was analyzed using the GEPIA (Gene Expression Profiling Interactive Analysis) program (FIG. 1A). In 275 tumor samples and 349 normal samples, only GLUT1 was significanμLy overexpressed in CRC, an observation that indicated that GLUT1 is a valid therapeutic target for CRC treatment. BAY-876 (FIG. 1B) was the first highly selective GLUT1 inhibitor that reduced glucose uptake and inhibited tumor growth. BAY-876 was tested using CRC cell line LS174T and patient-derived CRC cell line Pt2377 (38,39). BAY-876 inhibited the proliferation of these cell lines at nanomolar concentrations (FIG. 1C).

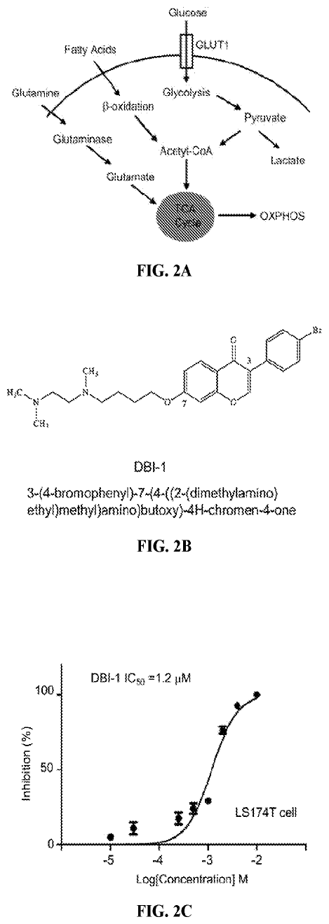

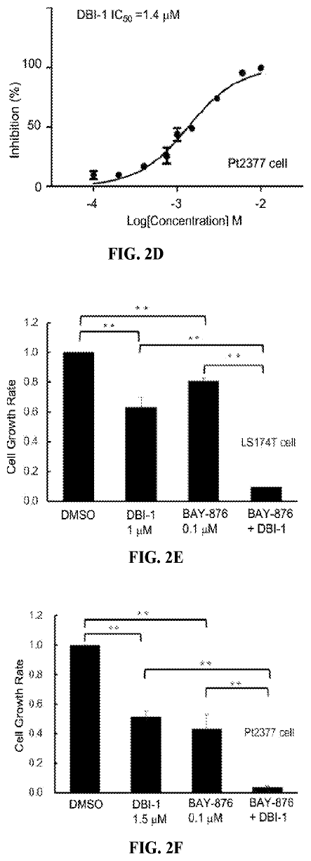

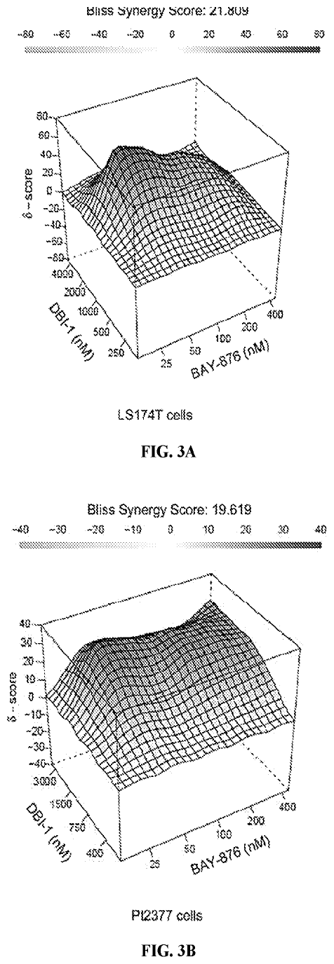

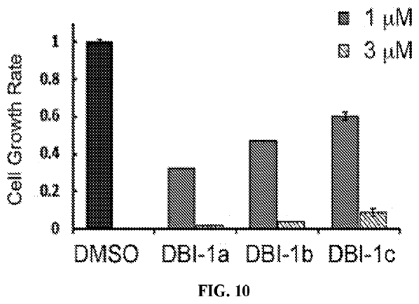

Identification of a semi-synthetic isoflavone that enhanced the efficacy of BAY-876 on CRC cell inhibition. Although glucose is a major energy source of ATP for cancer cells, cancer growth also depends on other energy sources, including amino and fatty acids. Most tumor cells, especially cancer stem cells (CSCs), rely on OXPHOS for ATP production (FIG. 2A). Since BAY-876 only blocked glucose uptake, cancer cells developed resistant mechanism by reprogramming energy metabolism pathways. In addition, BAY-876 is somewhat toxic to mice at effective doses (e.g., 4.5 mg/kg), a finding that suggested that a combination therapy should be used to reduced the minimal effective dose of BAY-876. A unique, semisynthetic natural compound library (53, 54) was screened, and a diaminobutoxy-substituted isoflavonoid was identified, namely 3-(4-bromophenyl)-7-(4-((2-(dimethylamino)ethyl) (methyl) amino) butoxy)-4H-chromen-4-one (hereinafter identified as DBI-1) (FIG. 2B) that inhibited the proliferation of LS174T and Pt2377 cells with inhibitory concentration IC₅₀ of 1.2 μM and 1.4 μM, respectively (FIGS. 2C-2D). Various DBI-1 analogs also inhibited the proliferation of LS174T CRC cells (FIG. 10). For both CRC cell lines, the combination of BAY-876 and DBI-1 significanμy inhibited cell proliferation (FIGS. 2E-2F). To confirm that the relationship between BAY-876 and DBI-1 was synergistic rather than additive, synergy scores were calculated using the SynergyFinder web application (Version 2.0) at default parameters with Bliss as the reference model. In LS174T and Pt2377 cells, the Synergy Score for the combination of BAY-876 and DBI-1 was 21.809 and 19.619, respectively, indicating a significant synergistic effect (i.e., value >10) (FIGS. 3A-3B).

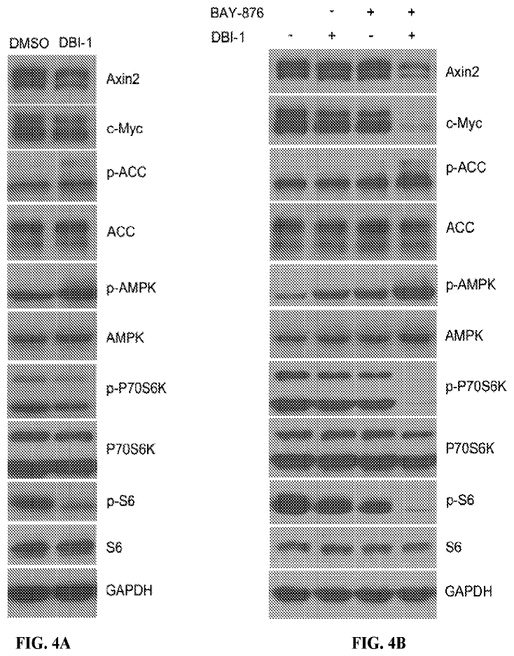

Synergistic effects of DBI-1 and BAY-876 on cell signaling pathways in CRC cells. To determine the mechanisms of DBI-1 and its combinational effects with BAY-876, their effects on cell signaling pathways was analyzed in LS174T cells. DBI-1 alone at a concentration of 3 μM inhibited the expression of Wnt target genes (Axin2 and c-Myc), activated the AMPK signaling (pAMPK and pACC), and inhibited the mTOR signaling (p70S6K and pS6) (FIG. 4A). When BAY-876 (1 μM) was used in combination with DBI-1 (3 μM), their effects were consistent (FIG. 4B) with their synergistic effects.

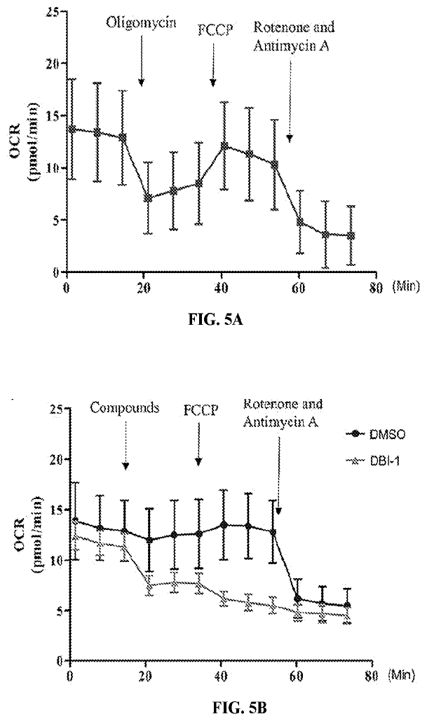

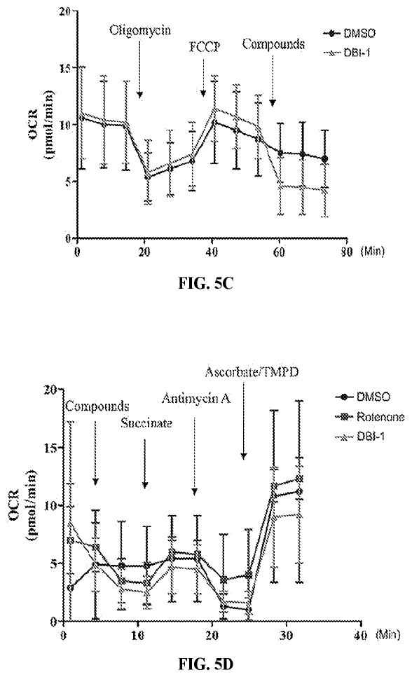

DBI-1 is a mitochondrial complex I inhibitor. Prior studies indicated that a number of mitochondrial uncouplers could inhibited Wnt signaling and activate AMPK signaling. To test if DBI-1 also regulated mitochondrial function, the effect of DBI-1 on oxidative phosphorylation was analyzed using a Seahorse assay. In the standard assay, the cells were sequencially treated with oligomycin, an inhibitor of mitochondrial complex V/ATP synthase; and with FCCP, a mitochondrial uncoupler; and with rotenone/antimycin, a mixture of complex I inhibitor and a complex III inhibitor. Oligomycin inhibits ATP-linked respiration and decreased OCR (oxygen consumption rate), followed by an increase in OCR upon addition of FCCP that induced proton leakage and dissociated the ETC from ATP synthesis. The FCCP-induced OCR could be further inhibited by complex I and complex III inhibitors (FIG. 5A). Oligomycin was first replaced with DBI-1. DBI-1 inhibited decreased OCR but the decrease was not rescued by FCCP, suggesting that DBI-1 is not a mitochondrial uncoupler or a complex V/ATP synthase inhibitor (FIG. 5B). Next, rotenone/antimycin A was replaced with DBI-1. DBI-1 inhibited the OCR to a similar extent as rotenone/antimycin (FIG. 5C), implying that it inhibited complex I or complex III of the ETC.

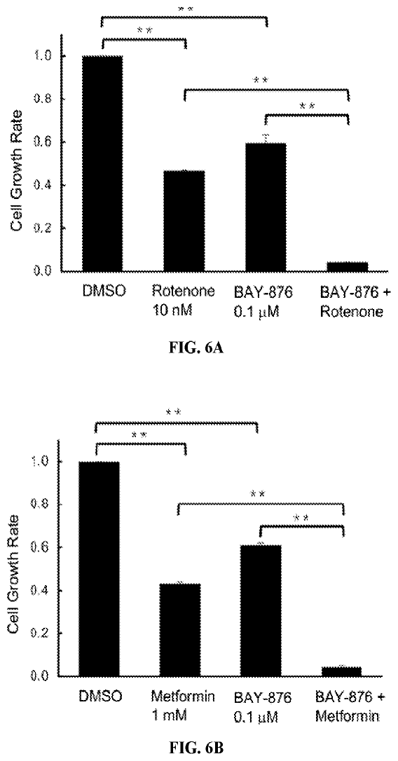

To reveal the exact mechanism by which DBI-1 affects mitochondria, substrate specific ETC/oxidative phosphorylation activity was measured using plasma membrane permeabilizer (PMP)-treated CRC cells with or without the addition of DBI-1 or known inhibitors of the ETC complex (FIG. 5D). Pyruvate, a complex I-linked substrate, was administered to permeabilized cells, followed by the addition of DMSO and either the complex I inhibitor rotenone or DBI-1. DBI-1 inhibited pyruvate/NADH-linked respiration relative to DMSO. This effect was similar to that of the complex I inhibitor, rotenone, a result that suggested that DBI-1 was also a complex I inhibitor. Next, succinate was added to drive respiration from succinate dehydrogenase (complex II) to direcμLy feed electrons into the ubiquinone pool, bypassing the inhibition of complex I. There was an increase in respiration in permeabilized cells with both rotenone treatment and DBI-1 treatment, a result that ruled out the possibility that DBI-1 inhibited the complexes II, III, and IV. a complex III inhibitor, antimycin A, was subsequently added, and OCR was inhibited. This inhibition was “rescued” by injection of the complex III electron donor, N,N,N,N-tetramethyl-p-phenylenediamine (TMPD), bypassing the complex III block and delivering electrons direcμLy to cytochrome c oxidase (complex IV). In conclusion, the results from Seahorse assays using ETC complex-specific inhibitors and PMP-permeabilized cells suggested that DBI-1 was a complex I inhibitor. This was consistent with previous findings that disruption of mitochondrial function activated AMPK, and inhibited Wnt signaling. To validate these findings, the combinational effects of BAY-876 were analyzed with two well-known complex I inhibitors, Rotenone and Metformin. Both compounds had synergistical effects with BAY-876 in inhibiting the proliferation of LS174T cells. These results suggest that other complex I inhibitors could also be used in combination with GLUT1 inhibitors for CRC treatment (FIGS. 6A-B).

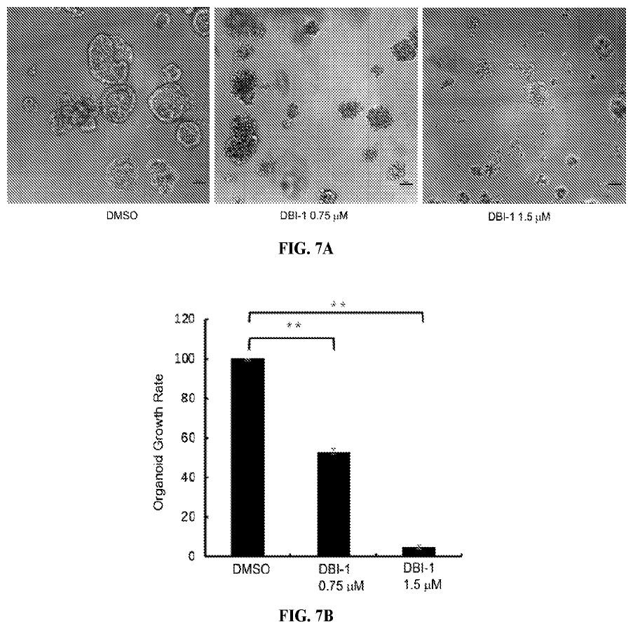

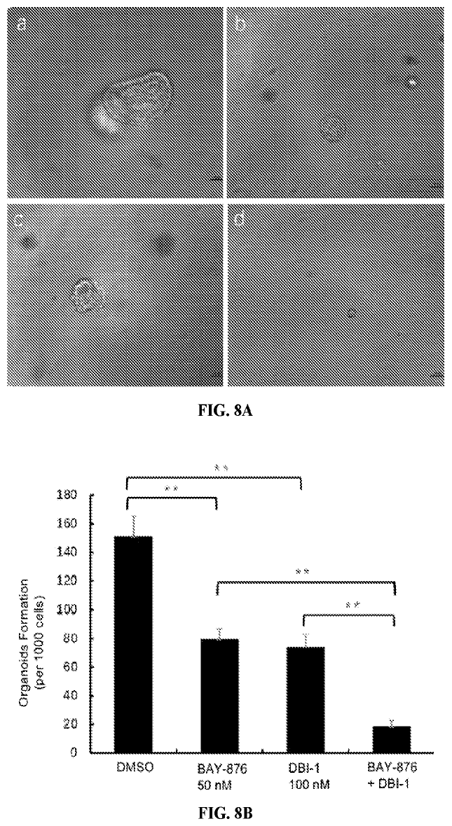

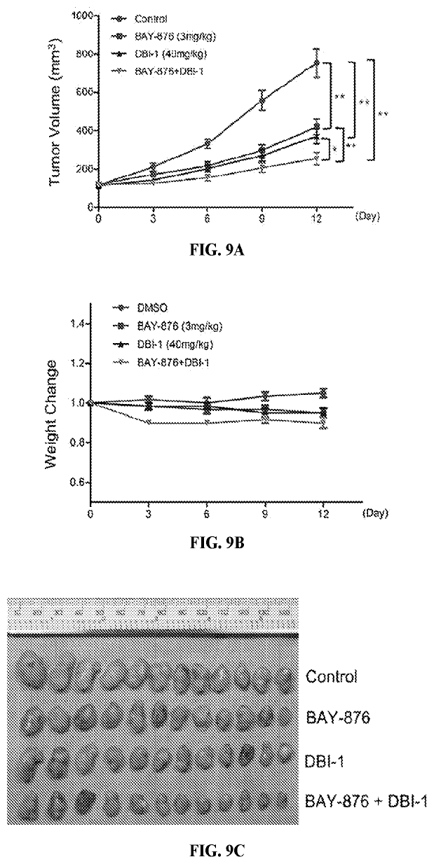

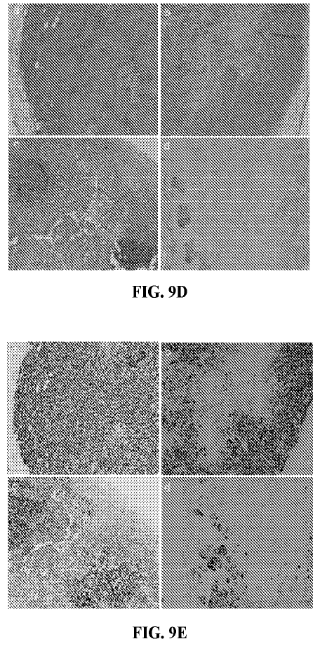

Combination of BAY-876 with DBI-1 inhibited CRC organoids and CRC xenografts in mouse models. The effects of DBI-1 were evaluated using a more pathologically relevant 3D tumor organoid model established from Apcf/+/KrasLSL-G12D/Vil-Cre compound mutant mice (40,41). DBI-1 inhibited organoid growth in a dose-dependent manner (FIGS. 7A-B). Furthermore, the combination of BAY-876 with DBI-1 significantly inhibited tumor organoid formation from single cells (FIGS. 8A-B) and prompted us to explore the in vivo therapeutic efficiencies of DBI-1 and its combination with GLUT-1 inhibitors in mouse models. To evaluate the in vivo tumor suppressive potential of DBI-1 and BAY-876, a LS174T CRC cell xenograft model was established in SCID mice. Daily administration of either BAY-876 at 3 mg/kg/day or DBI-1 at 40 mg/kg/day for two weeks inhibited tumor growth (FIGS. 9A and C). The combination treatment of BAY-876 and DBI-1 inhibited tumor growth more significanμy relative to either compound used alone (FIGS. 9A and C). No significant toxicity was found for either compound based on mice body weight growth rate (FIG. 9B). Tumor sections were analyzed with H&E and Ki-67 staining (FIGS. 9D-E). The cell proliferation marker ki-67 was significanμy reduced in the tumor samples treated with the combination of BAY-876 and DBI-1. In summary, DBI-1 and its isoflavonoid analogs (Tables 4 and 5) have potential utility as anticancer agents when used alone or in combination with other energy metabolism inhibitors, such as glucose transport inhibitors, glycolytic inhibitors, fatty acid oxidation inhibitors, or glutaminase inhibitors.

TABLE 4
Structure and Formula

| ID | Structure | Formula | MW |
|---|---|---|---|
| WZY-1-31 | 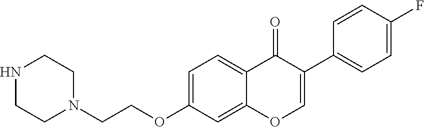 [SMILES: O=c1c(-c2ccc(F)cc2)coc2cc(OCCN3CCNCC3)ccc12] | C21H21FN2O3 | 368.41 |
| WZY-1-33 | 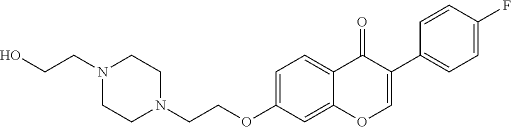 [SMILES: O=c1c(-c2ccc(F)cc2)coc2cc(OCCN3CCN(CCO)CC3)ccc12] | C23H25FN2O4 | 412.46 |
| WZY-1-43 | 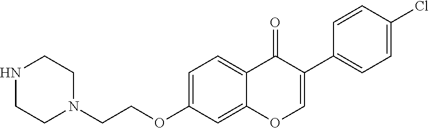 [SMILES: O=c1c(-c2ccc(Cl)cc2)coc2cc(OCCN3CCNCC3)ccc12] | C21H21ClN2O3 | 384.86 |
| WZY-1-45 | 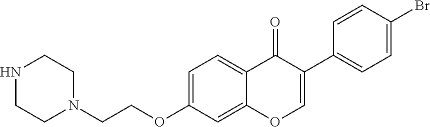 [SMILES: O=c1c(-c2ccc(Br)cc2)coc2cc(OCCN3CCNCC3)ccc12] | C21H21BrN2O3 | 429.31 |
| WZY-1-47 | 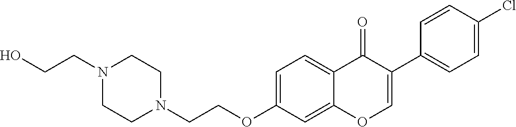 [SMILES: O=c1c(-c2ccc(Cl)cc2)coc2cc(OCCN3CCN(CCO)CC3)ccc12] | C23H25ClN2O4 | 428.91 |
| WZY-1-49 | 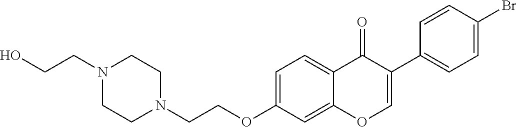 [SMILES: O=c1c(-c2ccc(Br)cc2)coc2cc(OCCN3CCN(CCO)CC3)ccc12] | C23H25BrN2O4 | 473.37 |
| WZY-1-59 | 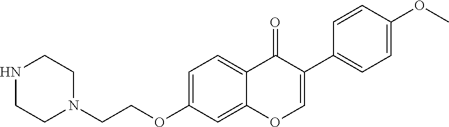 [SMILES: COc1ccc(-c2coc3cc(OCCN4CCNCC4)ccc3c2=O)cc1] | C22H24N2O4 | 380.44 |
| WZY-1-61 | 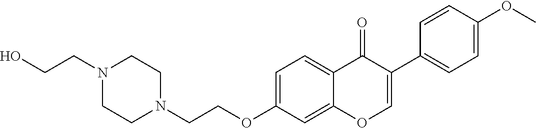 [SMILES: COc1ccc(-c2coc3cc(OCCN4CCN(CCO)CC4)ccc3c2=O)cc1] | C24H28N2O5 | 424.50 |
| WZY-1-67 | 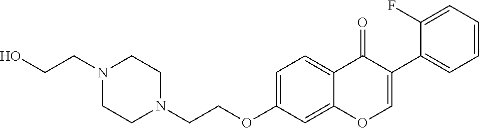 [SMILES: O=c1c(-c2ccccc2F)coc2cc(OCCN3CCN(CCO)CC3)ccc12] | C23H25FN2O4 | 412.46 |
| XDQ-1-9 | 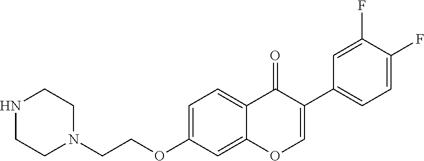 [SMILES: O=c1c(-c2ccc(F)c(F)c2)coc2cc(OCCN3CCNCC3)ccc12] | C21H20F2N2O3 | 386.14 |
| XDQ-1-13 | 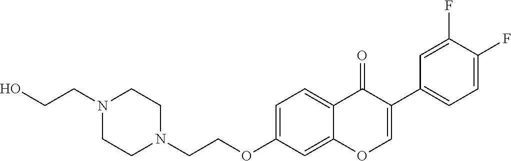 [SMILES: O=c1c(-c2ccc(F)c(F)c2)coc2cc(OCCN3CCN(CCO)CC3)ccc12] | C23H24F2N2O4 | 430.45 |
| XDQ-1-19 | 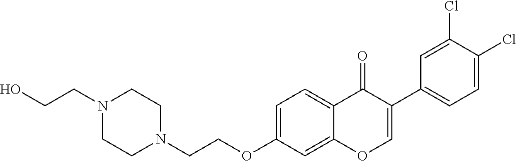 [SMILES: O=c1c(-c2ccc(Cl)c(Cl)c2)coc2cc(OCCN3CCN(CCO)CC3)ccc12] | C23H24Cl2N2O4 | 463.36 |
| XDQ-1-25 | 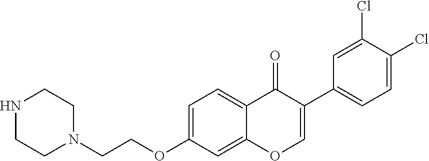 [SMILES: O=c1c(-c2ccc(Cl)c(Cl)c2)coc2cc(OCCN3CCNCC3)ccc12] | C21H20Cl2N2O3 | 419.3 |
| WZY-1-129 | 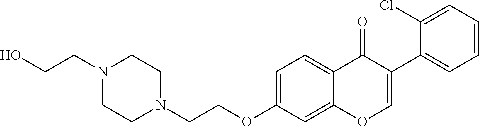 [SMILES: O=c1c(-c2ccccc2Cl)coc2cc(OCCN3CCN(CCO)CC3)ccc12] | C23H25ClN2O4 | 428.91 |
| WZY-1-77 | 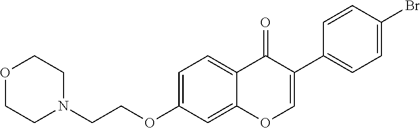 [SMILES: O=c1c(-c2ccc(Br)cc2)coc2cc(OCCN3CCOCC3)ccc12] | C21H20BrNO4 | 430.3 |
| WZY-1-91 | 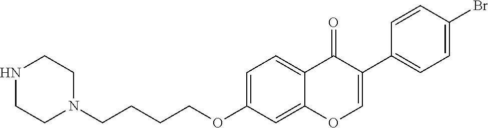 [SMILES: O=c1c(-c2ccc(Br)cc2)coc2cc(OCCCCN3CCNCC3)ccc12] | C23H25BrN2O3 | 457.37 |
| WZY-1-95 | 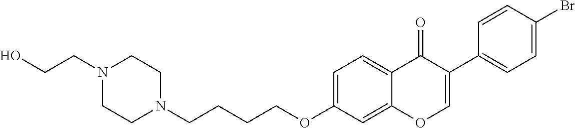 [SMILES: O=c1c(-c2ccc(Br)cc2)coc2cc(OCCCCN3CCN(CCO)CC3)ccc12] | C25H29BrN2O4 | 501.42 |
| WZY-1-97 | 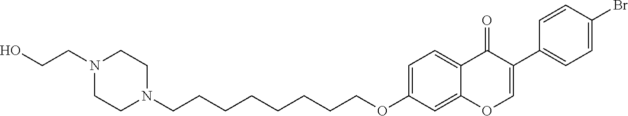 [SMILES: O=c1c(-c2ccc(Br)cc2)coc2cc(OCCCCCCCCN3CCN(CCO)CC3)ccc12] | C29H37BrN2O4 | 557.53 |
| WZY-1-101 | 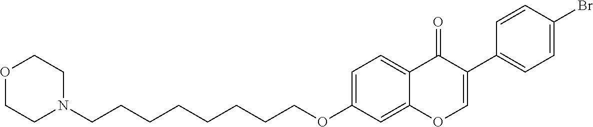 [SMILES: O=c1c(-c2ccc(Br)cc2)coc2cc(OCCCCCCCCN3CCOCC3)ccc12] | C27H32BrNO4 | 514.46 |
| WZY-1-103 | 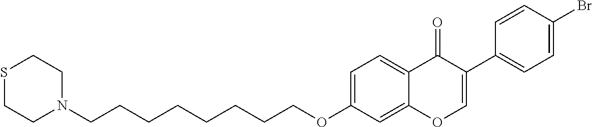 [SMILES: O=c1c(-c2ccc(Br)cc2)coc2cc(OCCCCCCCCN3CCSCC3)ccc12] | C27H32BrNO3S | 530.52 |
| WZY-1-113 | 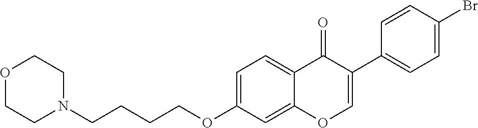 [SMILES: O=c1c(-c2ccc(Br)cc2)coc2cc(OCCCCN3CCOCC3)ccc12] | C23H24BrNO4 | 458.35 |
| WZY-1-123 | 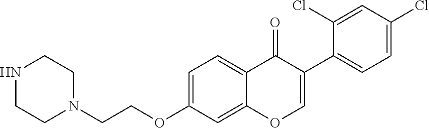 [SMILES: O=c1c(-c2ccc(Cl)cc2Cl)coc2cc(OCCN3CCNCC3)ccc12] | C21H20Cl2N2O3 | 419.3 |
| WZY-1-125 | 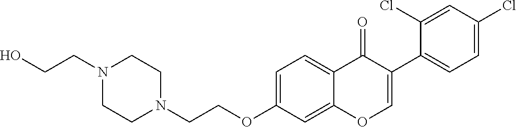 [SMILES: O=c1c(-c2ccc(Cl)cc2Cl)coc2cc(OCCN3CCN(CCO)CC3)ccc12] | C23H24Cl2N2O4 | 463.36 |
| WZY-1-131 | 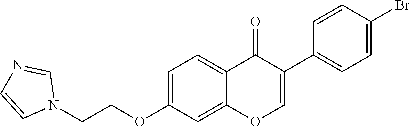 [SMILES: O=c1c(-c2ccc(Br)cc2)coc2cc(OCCn3ccnc3)ccc12] | C20H15BrN2O3 | 411.26 |
| WZY-1-139 | 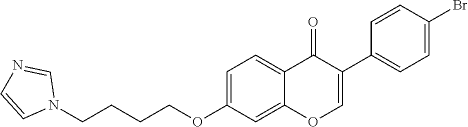 [SMILES: O=c1c(-c2ccc(Br)cc2)coc2cc(OCCCCn3ccnc3)ccc12] | C22H19BrN2O3 | 439.31 |
| WZY-1-141 | 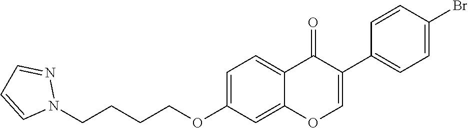 [SMILES: O=c1c(-c2ccc(Br)cc2)coc2cc(OCCCCn3cccn3)ccc12] | C22H19BrN2O3 | 439.31 |
| WZY-1-143 | 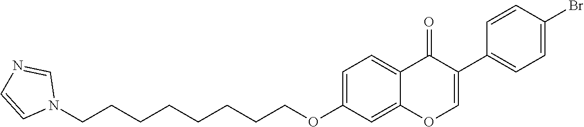 [SMILES: O=c1c(-c2ccc(Br)cc2)coc2cc(OCCCCCCCCn3ccnc3)ccc12] | C26H27BrN2O3 | 495.42 |
| WZY-1-149 | 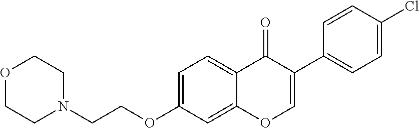 [SMILES: O=c1c(-c2ccc(Cl)cc2)coc2cc(OCCN3CCOCC3)ccc12] | C21H20ClNO4 | 385.84 |
| WZY-1-155 | 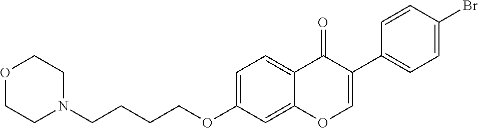 [SMILES: O=c1c(-c2ccc(Br)cc2)coc2cc(OCCCCN3CCOCC3)ccc12] | C23H24BrNO4 | 458.35 |
| WZY-1-193 (DBI-2) | 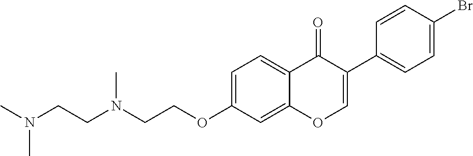 [SMILES: CN(C)CCN(C)CCOc1ccc2c(=O)c(-c3ccc(Br)cc3)coc2c1] | C22H25BrN2O3 | 445.36 |
| WZY-1-195 (DBI-1) | 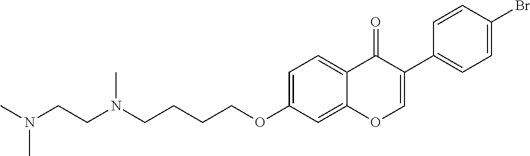 [SMILES: CN(C)CCN(C)CCCCOc1ccc2c(=O)c(-c3ccc(Br)cc3)coc2c1] | C25H29BrN2O4 | 473.41 |

TABLE 5
Inhibition Percent

| | HCT116 Cells | | | | |
|---|---|---|---|---|---|
| | Proliferation | | | OCV-8 Cell | |
| | Inhibition % | | | Inhibiton | |
| ID | 10 μM | 3 μM | 1 μM | 1 μM | 300 nM |
| WZY-1-31 | 89 | 0 | | | |
| WZY-1-33 | 35 | 0 | | | |
| WZY-1-43 | 93 | 92 | | 97 | 31 |
| WZY-1-45 | 93 | 94 | | 97 | 43 |
| WZY-1-47 | 94 | 50 | | | |
| WZY-1-49 | 94 | 80 | | | |
| WZY-1-59 | 92 | 0 | | | |
| WZY-1-61 | 52 | 0 | | | |
| WZY-1-67 | 0 | 0 | | | |
| XDQ-1-9 | 94 | 34 | | | |
| XDQ-1-13 | 61 | 10 | | | |
| XDQ-1-19 | 94 | 95 | | 99 | 60 |
| XDQ-1-25 | 92.5 | 93 | | 99 | 72 |
| WZY-1-129 | | 13 | | | |
| WZY-1-77 | | 61.4 | | | |
| WZY-1-91 | | 99.9 | | 94 | 34 |
| WZY-1-95 | | 99 | | 93 | 30 |
| WZY-1-97 | | 99.7 | | 96 | 39 |
| WZY-1-101 | | 50.6 | | | |
| WZY-1-103 | | 42.6 | | | |
| WZY-1-113 | | 62.7 | | | |
| WZY-1-123 | | 67.1 | | | |
| WZY-1-125 | | 43.1 | | | |
| WZY-1-131 | | 22 | | | |
| WZY-1-139 | | 58.7 | | | |
| WZY-1-141 | | 63.9 | | | |
| WZY-1-143 | | 61.2 | | | |
| WZY-1-149 | | 51.4 | | | |
| WZY-1-155 | | 65.3 | | | |
| WZY-1-193 | | | 26 | 87 | 57 |
| (DBI-2) | | | | | |
| WZY-1-195 | | | 45 | 96 | 57 |
| (DBI-1) | | | | | |

### Discussion

The Warburg effect is a recent focal point for the discovery of new therapeutic targets and cancer drugs. Due to complexity of ATP production, combination therapies that disrupt several branches of cellular energy metabolism may offer a better approach that just monotherapies. These combination strategies may also reduce the levels of key metabolites that could be used as building blocks or signaling molecules for tumor progression. In this study, a series of natural isoflavones and semisynthetic isoflavonoids were screened, and a diaminobutoxy-substituted isoflavonoid, DBI-1, was identified that had synergistic effects with GLUT1 inhibitor, BAY-876, on CRC cells in vitro and in vivo. Seahorse assays measuring oxygen consumption rate (OCR) in conjunction with various ETC inhibitors indicated that DBI-1 selectively inhibited complex. Combinations of BAY-876 and DBI-1 synergistically inhibited the proliferation of LS174T and Pt2377 CRC cells. In addition, DBI-1 and BAY-876 synergistically inhibited Wnt signaling and mTOR signaling and activated AMKP signaling in vitro and repressed tumor growth in vivo.

There are 14 reported isoforms of GLUT that are subdivided into three different protein classes based on their phylogenetic homology (65). Among these GLUT transporters, the class I isoforms, GLUT1-4, particularly GLUT1, were most intensively studied (14). Expression of GLUT1-4 genes was analyzed using the GEPIA program and found that only GLUT1 was significanμy overexpressed in CRC, an observation that suggested that GLUT1 could be either a biomarker or therapeutic target for CRC (FIG. 1A).

Despite promising in vitro and in vivo activity, none of the reported GLUT inhibitors proceeded to clinical human studies (66). Several GLUT1 inhibitors, including BAY-876, WZB117, NV-5440 and STF-31, had promising anti-cancer activities. BAY-876, for example, had the lowest reported _{IC50} (2 nM) for DLD-1 CRC cells (21,22,67-69). The efficacy of BAY-876 on different ovarian cancer cell lines displayed variable growth inhibition: _{IC50} of 60 nM for OVCAR-3 cells and 188 nM for SKOV-3 cells (23). In contrast, A2780 ovarian cancer cells were resistant to BAY-876 (23). In addition, BAY-876 did not inhibit the growth of triple-negative breast cancer (TNBC) cell lines, BT549, MDA-MB-436 and HCC70, at a concentration of 3 μM (19,70). BAY-876 inhibited the proliferation of CRC LS174T and Pt2377 cells at nanomolar concentrations (FIG. 1C), a finding that suggested that GLUT1 was a promising target for CRC.

Cancer cells have a high degree of metabolic plasticity and generate energy from a variety of sources (27). This observation suggests that targeting multiple energy producing pathways would be an effective strategy for overcoming drug resistance. For example, 2-DG is a glucose analogue that is unable to undergo glycolysis. The glutaminolysis inhibitor aminooxyacetate (AOA) synergizes with 2-DG to decrease cell proliferation in ovarian cancer cells, an outcome that indicated that dual inhibition of glycolysis and glutaminolysis was a valid therapeutic strategy for the treatment of ovarian cancer (71). β-Oxidation has been described as one of the essential energy sources for triple-negative breast cancers (72). A combination treatment of glutaminase inhibitor CB-839 and the carnitine palmitoyltransferase 1 (CPT1) inhibitor, etomoxir, inhibited the glutaminase inhibition-resistant breast cancer cell line HCC1937. In addition, this combination decreased cell migration in the HCC1937 cells, an observation that indicated that dual targeting of glutaminase and CPT1 activities had therapeutic relevance for treating TNBCs (73).

Recently, a promising complex I inhibitor, IACS-010759, was developed and progressed into phase I clinical trials. In preclinical models, 5-10 mg/kg/day IACS-010759 resulted in tumor regression. However, IACS-010759 was not tolerated at 25 mg/kg/day (28). To improve the therapeutic window of complex I inhibitors, it is important to identify biomarkers associated with drug sensitivity. Tumor cells with deficiency in glycolysis or low aspartate levels are particularly sensitive to complex I inhibitors (75,78). Complex I inhibitors also selectively inhibit Pten-null cells (74). Combination therapies based on these mechanisms may also improve the therapeutic window. In this study, the combination of a GLUT1 inhibitor and mitochondrial complex I OXPHOS inhibitor provided a promising approach for CRC treatment. DBI-1 represents a new pharmacophore for developing mitochondrial complex I inhibitors for future clinical studies in which combinatorial therapies hold promise.

### Example 2

### Materials and Methods

General Chemistry Methods. Chemicals and solvents were obtained from Sigma Aldrich (St. Louis, MO, USA) with the exception of resorcinol and 4-bromophenylacetic acid (Acros Organic, Carlsbad, CA, USA), 1,4-dibromobutane (Oakwood Chemical, Estill, SC, USA), 1,8-Dibromooctane (Rhawn, Shanghai, China), sodium iodide (Fisher Scientific, Waltham, MA, USA), Piperazine,1-(2-Hydroxyethyl)piperazine, (J&K Scientific, Shijiazhuang, Hebei, China). Nuclear magnetic resonance spectra were determined in CDCl3, DMSO-d6 using Bruker instruments (1H, 600; 13C, 151 Mz; Bruker, Inc., Billerica, Ma, USA). High resolution electrospray ionization (ESI) mass spectra were recorded on a LTQ-Orbitrap Velos mass spectrometer (Thermo Fisher Scientific, Waltham, MA, USA). The FT resolution was set at 100,000 (at 400 m/z). Samples were introduced through direct infusion using a syringe pump with a flow rate of 5 μL/min. Purity was established by combustion analyses performed by AμLantic Microlabs, Inc. (Norcross, GA, USA).

### General Procedure a

To a mixture of resorcinol (15 mmol, 1 equiv.) and the corresponding phenylacetic acid (16.5 mmol, 1.1 equiv.) was added boron tribromide etherate (120 mmol, 8 equiv.). The mixture was heated at 85° C. for 4 h under a nitrogen atmosphere. The solution was cooled to 0° C., and DMF (15 mL) was added dropwise slowly. In another flask of adding DMF (20 mL) was slowly added phosphorous pentachloride (22.5 mmol, 1.5 equiv.) at 0° C., then stirring this mixture for 30 min at room temperature and heating it for 30 min at 55° C., blending it with the mixtures of former flask. This blended reaction was stirred for 3 h at room temperature then poured it into boiling aqueous hydrochloric acid (120 mL, 0.1M). There was solid precipitation as the mixture was allowed to cool to room temperature, filtered it and washed with water. The crude product was dried under reduced pressure overnight, then purified via chromatography on SiO2 (solvent system: ethyl acetate:hexanes-1:6) to afford desire products 3,4.

### General Procedure b

To the mixture of the corresponding production of general procedure a (7.52 mmol, 1 quiv.) and potassium carbonate (18.8 mmol 2.5 equiv.) was added DMF (40 ml) and dibromo alkane (1.88 mmol, 5 equiv.). The reaction was heated for 3 h at 80° C. under the nitrogen protection. Then mixture was poured into the water (100 ml) and afford solid precipitate. filtered it and washed with water. The crude product was dried under reduced pressure overnight, then purified via chromatography on SiO2 (solvent system: ethyl acetate:hexanes-1:2) to afford desire products 5,6,7,8.

### General Procedure c

To the mixture of the corresponding production of general procedure b (2 mmol, 1 quiv.) and sodium iodide (2.4 mmol, 1.2 quiv.) was added DMF (18 ml), amine (2.4 mmol, 1.2 equiv.) and DIPEA (7 mmol, 3.5 equiv.). The reaction was heated for 3 h at 60° C. under the nitrogen protection, then quenched with water (50 ml) and extracted three times with ethyl acetate (50 ml). The organic layer was collected and dried with Magnesium Sulfate, filtered it and concentrated by evaporator. Then purified via chromatography on SiO2 (solvent system: Ammonia:methanol:methylene chloride-1:10:100,) to afford desire products DBI-1, DBI-2, DBI-3, DBI-4, DBI-5, DBI-6, 9.

3-(4-bromophenyl)-7-hydroxy-4H-chromen-4-one. (3) Following general procedure a, 3 was obtained from 1 in 39.4% yield.

3-(3,4-dichlorophenyl)-7-hydroxy-4H-chromen-4-one. (4) Following general procedure a, 4 was obtained from 2 in 24.1% yield.

7-(2-bromoethoxy)-3-(4-bromophenyl)-4H-chromen-4-one. (5) Following general procedure b, 5 was obtained from 3 and 1,2-dibromoethane.

7-(4-bromobutoxy)-3-(4-bromophenyl)-4H-chromen-4-one. (6) Following general procedure b, 6 was obtained from 4 and 1,4-dibromobutane in 44% yield.

7-((8-bromooctyl)oxy)-3-(4-bromophenyl)-4H-chromen-4-one. (7) Following general procedure b, 7 was obtained from 4 and 1,8-dibromooctane in ?yield.

7-(2-bromoethoxy)-3-(3,4-dichlorophenyl)-4H-chromen-4-one. (8) Following general procedure b, 8 was obtained from 4 and 1,2-dibromoethane in 31.8% yield.

3-(4-bromophenyl)-7-(2-((2-(dimethylamino)ethyl)(methyl)amino)ethoxy)-4H-chromen-4-one. (DBI-1) Following general procedure c, DBI-1 was obtained from 6 and N1,N1,N2-trimethylethane-1,2-diamine in 36.0% yield.

3-(4-bromophenyl)-7-((8-(4-(2-hydroxyethyl)piperazin-1-yl)octyl)oxy)-4H-chromen-4-one. (DBI-2) Following general procedure c, DBI-2 was obtained from 7 and 2-(piperazin-1-yl)ethan-1-ol in 66.0% yield.

### 3-(4-bromophenyl)-7-(4-(piperazin-1-yl)butoxy)-4H-chromen-4-one. (DBI-3)

Following general procedure c, DBI-3 was obtained from 6 and piperazine in 45.0% yield.

3-(4-bromophenyl)-7-(2-(piperazin-1-yl)ethoxy)-4H-chromen-4-one. (DBI-4) Following general procedure c, DBI-4 was obtained from 5 and piperazine in 57.0% yield.

3-(3,4-dichlorophenyl)-7-(2-(piperazin-1-yl)ethoxy)-4H-chromen-4-one. (DBI-5) Following general procedure c, DBI-5 was obtained from 8 and piperazine in 19.0% yield.

3-(3,4-dichlorophenyl)-7-(2-(4-(2-hydroxyethyl)piperazin-1-yl)ethoxy)-4H-chromen-4-one. (DBI-6) Following general procedure c, DBI-6 was obtained from 8 and 2-(piperazin-1-yl)ethan-1-ol in 20.0% yield.

Ethyl 2-(4-(2-((3-(3,4-dichlorophenyl)-4-oxo-4H-chromen-7-yl)oxy)ethyl)piperazin-1-yl)acetate. (9) Following general procedure c, 9 was obtained from 8 and ethyl 2-(piperazin-1-yl)acetate in 41.1% yield.

2-(4-(2-((3-(3,4-dichlorophenyl)-4-oxo-4H-chromen-7-yl)oxy)ethyl)piperazin-1-yl)acetic acid. (DBI-7) Put 50 mg of 9 into HCl (PH=1), stirred at 950 for 4 h, cooled and filtered the precipitate, washed with a litμLe of water, dichloromethane, and methanol, respectively, and then dried under vacuum. DBI-7 was obtained in 52.9% yield.

### Biology Materials

Antibodies used in Western Blotting: Axin2 (#2151, Cell signaling technology), c-Myc (#1472-1, Epitomics), p-ACC (#11818, Cell signaling technology), ACC (#3676, Cell signaling technology), p-AMPK (#2535, Cell signaling technology), AMPK (#2532, Cell signaling technology), p-P70S6K (#9234, Cell signaling technology), P70S6K (#2708, Cell signaling technology), p-S6 (#4858, Cell signaling technology), S6 (#2317, Cell signaling technology), GAPDH (#GTX627408, GeneTex), Ki67 (#9449, Cell signaling technology).

The following Compound were used: BAY-876 (i.e., N4-[1-(4-cyanobenzyl)-5-methyl-3-(trifluoromethyl)-1H-pyrazol-4-yl]-7-fluoroquinoline-2,4-dicarboxamide) was acquired from Medkoo Bioscience (#530485).

Diet: The ketogenic diet (Cat No. TP201450) was purchased from Trophic Animal Feed High-tech Co., Ltd., China which is comparable to Bio-Serv F3666.

### Cell Lines and Cell Culture

LS174T-TR4 cells were obtained from Professor Hans Clevers and Marc van de Wetering at Utrecht University, and were selected with resistance to blasticidin (16). LS174T cells were cultured in RPMI1640 (Sigma, R8758) containing 5% Fetal Bovine Serum (Sigma F0926). HCT116 cells were purchased from ATCC and were cultured in DMEM (Sigma, D6429) containing 10% Fetal Bovine Serum (Sigma F0926). All cells were cultured at 37° C. with 5% CO₂ atmosphere in a water jacketed incubator (NuAire, Plymouth, MN).

### Western Blotting

Western blotting was performed as previously reported procedures (41). Cells were split into 6-well plates and incubated for 24 hours. Either DMSO or compounds dissolved in DMSO at indicated concentrations were added to each well for another 24 hours. Cells were lysed in 500 μL of lysis buffer containing 50 mmol/L HEPES, 100 mmol/L NaCl, 2 mmol/L EDTA, 1% (v/v) glycerol, 50 mmol/L NaF, 1 mmol/L Na₃VO₄, 1% (v/v) Triton X-100, and protease inhibitors (Sigma, P8340). Cell lysates were centrifuged for 10 minutes (18,000×g), and the supernatants were mixed with 6×protein loading buffer and boiled for 3 minutes. The samples were analyzed by standard Western blotting methods using the appropriate antibodies.

### Seahorse Assay for Oxygen Consumption Rate Measurement

The Seahorse assays were performed in compliance with a pre-announced procedure (41). Oxygen consumption was measured using the Seahorse XFe96 Extracellular Flow Analyzer (Agilent Technologies, Santa Clara, CA, USA). 2.5×10⁴ LS174T cells were seeded in XF96 Cell Culture microplate in 80 μL culture medium and incubated overnight. On the following day, the cell culture media were exchanged with Seahorse XF modified media. 1.0 μmol/L oligomycin, 1.0 μmol/L FCCP, and a mixture of 1.0 μmol/L rotenone and 1.0 μmol/L antimycin A were sequentially added during the measurements. To identify whether the compounds were ETC complex V/ATP synthase inhibitors, oligomycin was substituted with an equal volume of DMSO or compounds in DMSO solution. To determine whether compounds inhibited ETC complex I or III, rotenone and antimycin A were replaced with an equal volume of DMSO, or compounds dissolved in DMSO.

### Measurements of Mitochondrial ETC Complex Activity

Mitochondrial ETC complex activity was measured using the Agilent Seahorse Assay with XF permeabilizer (PMP; Agilent) in compliance with the manufacturer's protocol. Cells were genμLy washed with mitochondrial assay solution (MSA) containing 220 mmol/L mannitol, 70 mmol/L sucrose, 10 mmol/L KH₂PO₄, 5 mmol/L MgCl₂, 2 mmol/L HEPES, 1 mmol/L EGTA, and 0.2% (w/v) fatty acid free BSA. Then cell culture media was replaced with MAS supplemented with 4 mmol/L adenosine diphosphate (ADP), 10 mmol/L pyruvate, and 1 nmol/L PMP. Following calibration, the baseline OCR was measured with cycles of 0.5-minute mixing, 0.5-minute waiting, and 2-minute measuring to perform the test. After the baseline definition, the sequential injection of DMSO, test compounds or 2 μmol/L rotenone, 10 μmol/L succinate, 2 μmol/L antimycin A, and a combination of 10 mmol/L ascorbate and 100 μmol/L N,N,N,N-tetramethyl-p-phenylenediamine (TMPD) were performed. Data were then normalized on cell density using Biotek Cytation 1 (BioTek Instruments, Winooski, VT, USA).

### Cell Proliferation Inhibition Assay

Cell proliferation inhibition assay was conducted according to a previous report (48). The cells were split into 12-well plates at a density of 40,000 cells per well in 1 mL of culture medium and were cultured overnight at 37° C. Either 1 μL of the compounds dissolved at 10 mmol/L in DMSO or the vehicle control (DMSO) was added to the cells. After 5 days, removed the medium, and 200 μL of 0.25% trypsin was added. The cells were resuspended in phosphate-buffered saline (PBS) and counted with Vi-CELL XR 2.03 (Beckman Coulter, Inc. USA). The ratio R of the number of viable cells in the compound treatment group to the number of viable cells in the DMSO treatment group was defined as the relative growth rate, and the growth inhibition rate was calculated as (1−R)*100.

### Calculation of Synergy Scores of BAY-876 and DBI-6

The cell proliferation inhibition assay described above was conducted using 24-well plates on LS174T and HCT116 cells to assess the inhibitory effect of BAY-876 with or without DBI-6 at predetermined concentrations. The final treatment concentrations of BAY-876 were 0, 10, 30, 100, 300 nmol/L in the presence or absence of DBI-6 at 0, 100, 300, 1000, 3000 nmol/L in LS174T cells. The final treatment concentrations for BAY-876 were 0, 50, 100, 200, 400 nmol/L and for DBI-6 were 0, 100, 200, 400 and 800 nmol/L. Synergy scores were measured using Bliss as the reference model in the Synergyfinder web application (version 2.0; www.synergyfinder.org) with default parameters (49).

### Quantification of Apoptotic Cells and Necrotic Cells by Flow Cytometry

LS174T cells were cultured in 6-well plates at 37° C. for 24 hours. 3 μmol/L DBI-6 or 1 μmol/L BAY-876 were added to the cells and incubated for 24 hours. Cells of combination therapy group were treated with both DBI-6 and BAY-876. Then cells were harvested and washed with cold PBS twice and resuspended in 500 μL Binding Buffer. SubsequenμLy, the cells were stained with Annexin V-FITC and propidium iodide (PI) and incubated in dark for 5 minutes. The percentages of apoptosis and necrosis were determined by flow cytometry (BeckmanCoulter, Brea, CA, USA).

### RNA-Seq Transcriptomic Assay

LS174T cells were grown in 6-well plates for 24 hours and treated with 5 μmol/L DBI-6 for another 24 hours. The treated cells were harvested and the total RNA was extracted according to the manufacturer's instructions of the Eastep® Super Total RNA extraction kit (Promega (Beijing) Biotechnology Co., Ltd, Beijing, China). The DMSO treated LS174T cells were used as control, and there were three repetitions in both groups. The samples were submitted to BGI Genomics Co., Ltd (Shenzhen, Guangdong, China) for RNA-seq. Functional enrichment analysis of Gene ontology (GO) and Kyoto Encyclopedia of Genes and Genomes (KEGG) pathway were performed. The phyper function in R software was used to perform the enrichment analysis, calculate the Pvalue, and the Qvalue was obtained by correction of P-value. Generally, the function with Qvalue ≤0.05 was considered as significant enrichment.

### In Vivo Evaluation of Antitumor Effects in LS174T Xenografts

Rag1 mice of 6 to 8 weeks of age were kindly provided by Institute of Genetics and Developmental Biology, Chinese Academy of sciences. LS174T cells in exponential growth phase were harvested and mixed with an equal volume of Matrigel (Corning, Glendale, Arizona). Each mouse was subcutaneously injected on the lower flanks with 2×10⁶ cells. Tumor cell-injected mice were randomly divided into control and treatment groups when tumors were established and reached an average volume of 100 mm³ (3 male and 3 female mice/group, two tumors on each mouse).

After tumors were established, DBI-6 was formulated in a mixture of ethanol (5%), 20% solutol and 20% Cremophor EL (50%) and Citric acid (45%; 50 mmol/L, pH4.5) and was daily intraperitoneally administered to the DBI-6 group at dose of 40 mg/kg mouse body weight. BAY-876 was dissolved in DMSO (10%), PEG400 (25%), Tween 80 (5%) and PBS (60%) and was given to the mice by gavage feeding to the BAY-876 group at a daily dose of 3 mg/kg mouse body weight. The combination therapy group was daily treated with both DBI-6 at dose of 40 mg/kg and BAY-876 at the dose of 3 mg/kg mouse body weight. All mice of the control group, DBI-6 treatment group, BAY-876 treatment group and the combination therapy group were received the standard diet ad libitum for the duration of the study. There were two groups were fed the ketogenic diet (KD), in addition to this, the KD-fed combined with DBI-6 treatment group was daily treated with DBI-6 at dose of 40 mg/kg mouse body weight. The percent nutritional of KD is as follows: 3.2% of Cal from carbohydrates, 8.9% of Cal from protein, and 74.2% of Cal from fat. The food was changed daily.

The tumor sizes and mouse weights were measured every three days with calipers, and the tumor volume (Length×width²/₂) were calculated. The experiment was stopped at day 12 and mice were sacrificed. Tumors of all groups were isolated and fixed in 10% neutral buffered formalin and embedded in paraffin. Then the tumor sections were analyzed with H&E and Ki-67 staining. Plasma was collected and analyzed with the assay kit (Jiancheng Bioengineering Institute, Nanjing, China) according to the manufacturer's protocol.

### Hematoxylin and Eosin (H&E) Staining and Immunohistochemistry

Six (6) μm sections were collected and dried for 2 h at 60° C. Slides were deparaffinized and hydrated in xylene and graded ethanol solutions and washed two times in dH2O. For H&E staining, the slides were placed in Hematoxylin (Biosharp) for 3 minutes, washed with tap water and differentiated in 1% hydrochloride in 70% ethanol for 30 s. After differentiation, slides were rinsed in tap water then incubated in Eosin solution (Biosharp) for 5 min. Finally, the slides were dehydrated in graded ethanol and xylene. For immunofluorescence, the slides were heated in a microwave submersed in 0.01 mol/L citrate unmasking solution for antigen unmasking. After incubated in 3% hydrogen peroxide for 10 minutes, slides were blocked for 15 minutes at 37° C. and treated with Ki-67 primary antibody overnight at 4° C. The next day, secondary antibodies were added to slides for 15 minutes at 37° C. DAB substrate reaction was completed followed by Streptavidin-Biotin reaction. Hematoxylin was used for staining the nucleus.

### Statistics

Major biological assays were repeated at least twice. For the animal study, six mice with two tumors on the lower flanks of each mouse were used in each group. Data were expressed as mean±SEM and statistical analysis was performed with GraphPad Prism 5. The Student's two-tail t test was used to calculate the statistical significance among groups, and P<0.05 was defined statistically significant. Statistical significance was represented by * if P<0.05 and ** if P<0.01.

### Results

### SAR Study of DBI-1 Analogs.

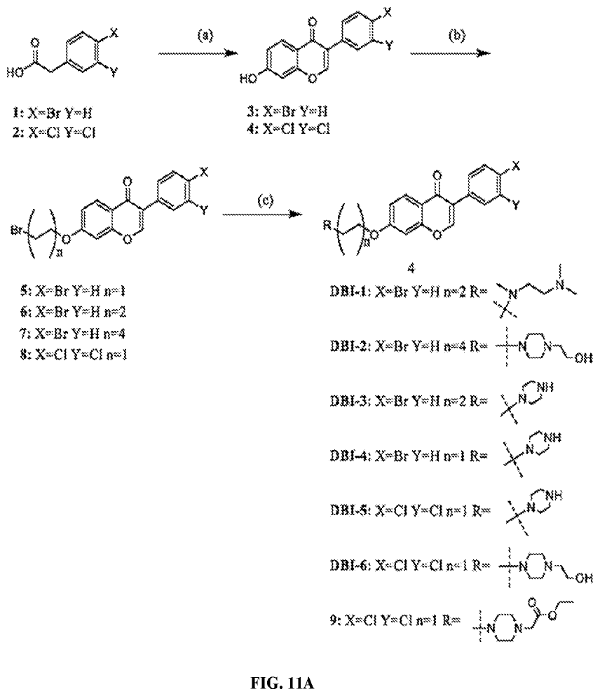

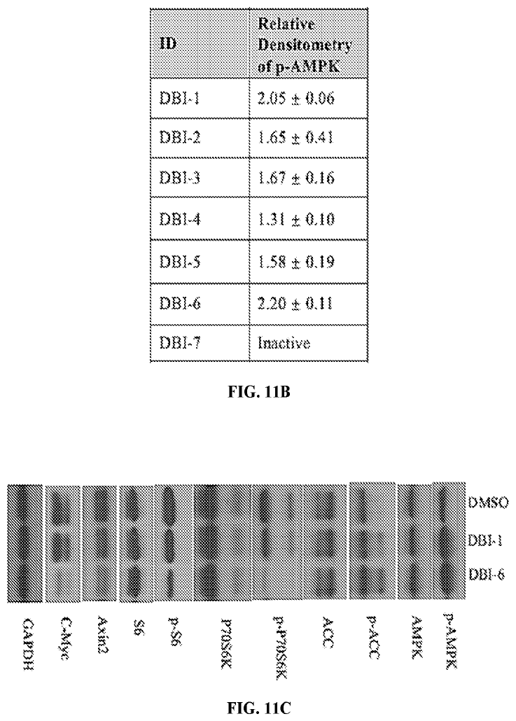

A diaminobutoxy-substituted isoflavonoid (DBI-1) was identified as a novel mitochondrial complex I inhibitor (90). DBI-1 reduced ETC-dependent energy production and activated amp-activated protein kinase (AMPK), a crucial energy sensor in the cells. Using AMPK phosphorylation assay as a readout, >30 DBI-1 analogs were synthesized and analyzed. Representative analogs were demonstrated in FIG. 11A. At 3 μM, DBI-1-6 but not DBI-7 induced AMPK phosphorylation (activation) in LS174T CRC cells (FIG. 11B). DBI-1 and DBI-6 had the best activity in AMPK activation in CRC cells. It has been reported that reducing ATP production by OXPHOS inhibitors or mitochondrial uncouplers not only activated AMPK signaling pathway, but also inhibited mTOR and Wnt signaling pathways in CRC cells (50,57,90). The effects of DBI-1 and DBI-6 on cell signaling pathways were compared in LS174T cells. DBI-1 or DBI-6 alone at 3 μM activated AMPK signaling (pAMPK and pACC) and inhibited mTOR signaling (p70S6K and pS6). Additionally, DBI-1 or DBI-6 inhibited the expression of Axin2 and c-Myc, major target genes of Wnt signaling. DBI-6 was more active than DBI-1 in these assays (FIG. 11C).

### DBI-6 Inhibited Mitochondrial Complex I.

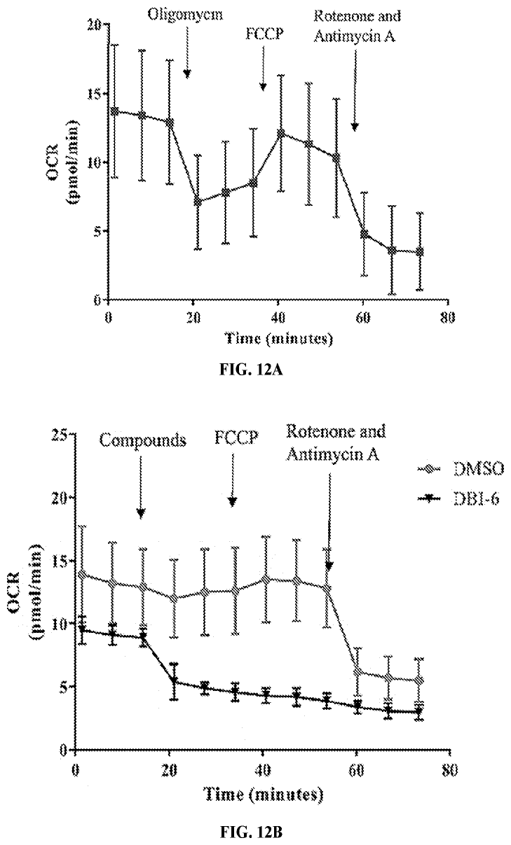

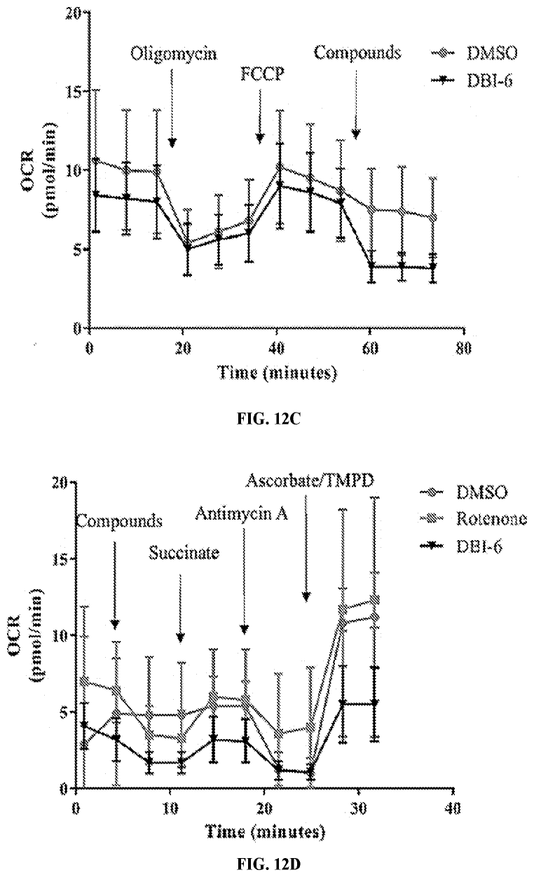

DBI-1 is a mitochondrial complex I inhibitor. To determine whether DBI-6 also plays a role in regulating mitochondrial function, the Seahorse assays were performed to investigate the effect of DBI-6 on oxygen consumption rate (OCR). In the standard assay, oligomycin, an inhibitor of mitochondrial complex V/ATP synthase, decreased OCR. Mitochondrial uncoupler, FCCP, rescued oligomycin-inhibited OCR. Then a mixture of complex I/III inhibitors, rotenone/antimycin A, inhibited the FCCP-induced OCR (FIG. 12A). Oligomycin was replaced with DBI-6 in the standard assay. OCR was inhibited but the decreased OCR could not be rescued by FCCP (FIG. 12B). This result indicated that DBI-6 is not a mitochondrial uncoupler or a complex V/ATP synthase inhibitor. Next, rotenone/antimycin A was replaced with DBI-6 and noticed that DBI-6 suppressed OCR in a similar manner to rotenone/antimycin A (FIG. 12C), suggesting that DBI-6 inhibited complex I and/or complex III of the ETC.

To determine whether DBI-6 inhibits complex I or complex III, substrate specific OXPHOS activity was measured using plasma membrane permeabilizer (PMP) treated CRC cells (FIG. 12D). The plasma membrane permeabilizer, derived from Clostridium perfringens, is a recombinant, mutant form of cholesterol-dependent cytolysin that oligomerizes to form pores that selectively permeate the cytoplasmic membrane, allowing the passage of solutes and large proteins of 200 kDa (91-93). The permeabilized cells were treated with pyruvate, a complex I-linked substrate. DMSO, complex I inhibitor, rotenone, or DBI-6 were subsequenμLy added. DBI-6 reduced pyruvate/NADH-linked respiration. This result was comparable to that of the complex I inhibitor rotenone. Next, succinate was added to drive respiration of succinate dehydrogenase (complex II), which sends electrons direcμLy into the ubiquinone pool while bypassing complex I. Respiration increased in both rotenone-treated and DBI-6-treated permeabilized cells, a fact excluded the possibility that DBI-6 was an inhibitor of complexes II, III, and IV. Finally, a complex III inhibitor, antimycin A, was added to the system and OCR was again suppressed. However, this suppressed was “rescued” by the injection of the complex III electron donor TMPD, which bypasses the complex III block and delivers electrons direcμLy to cytochrome c oxidase (complex IV). In summary, the Seahorse OCR assay data using ETC complex-specific inhibitors and PMP-permeabilized cells confirmed that DBI-6 is a complex I inhibitor that disrupts mitochondrial function.

### DBI-6 and GLUT1 Inhibitor, BAY-876, Synergistically Inhibited CRC Cells In Vitro.

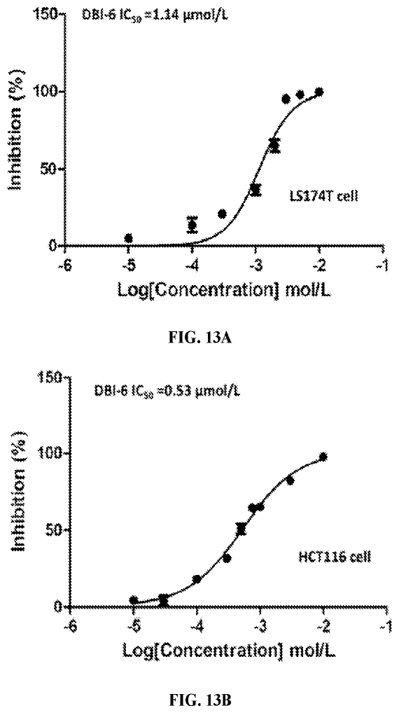

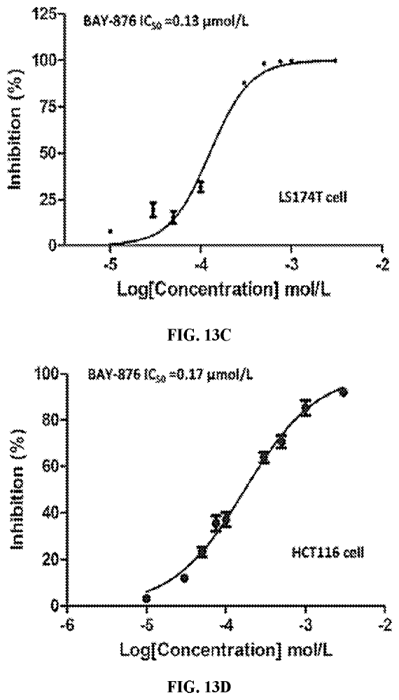

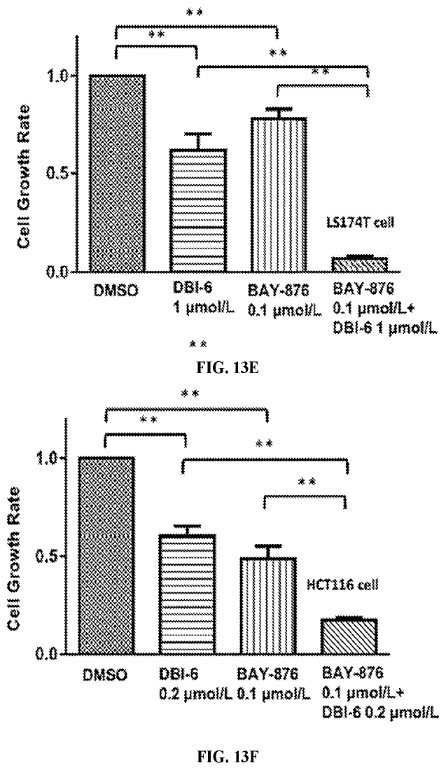

It has been reported that complex I inhibitors, including rotenone, metformin and DBI-1, had synergistic effects with GLUT1 inhibitor, BAY-876, on CRC cell inhibition (90). It was contemplated that DBI-6 should also has synergistic effects with BAY-876. DBI-6 inhibited the proliferation of LS174T and HCT116 CRC cells with IC₅₀ of 1.14 and 0.53 μM, respectively (FIGS. 13A and 13B). And BAY-876 inhibited the proliferation of LS174T and HCT116 CRC cells with IC₅₀ of 0.13 and 0.17 μM, respectively (FIGS. 13C and 13D). Both CRC cell lines, the combination of BAY-876 and DBI-6 dramatically inhibited cell proliferation (FIGS. 13E and 13F).

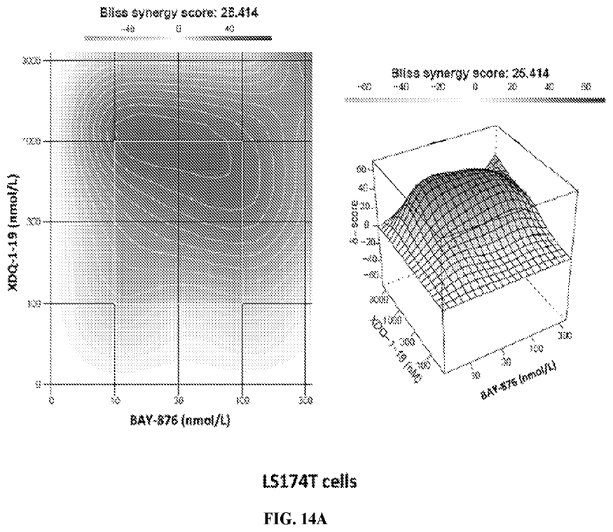

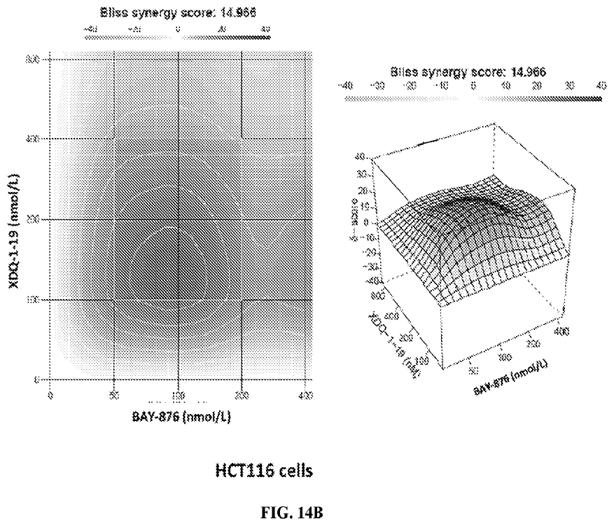

To verify that the combinational effects of BAY-876 and DBI-6 are synergistic rather than additive, the Bliss Synergy Scores was analyzed using the SynergyFinder program (version 2.0) (49). In LS174T cells, the synergy score for the combination of DBI-6 and BAY-876 was 25.4 at certain concentrations (DBI-6 0.1-3 μM and BAY-876 10-300 nM). In HCT116 cells, the synergy score was 14.966 for the combination of DBI-6 at 100-800 nM and BAY-876 at 50-400 nM. These data indicated a significant synergistic effect. (i.e., synergy score values >10; FIGS. 14A and 14B).

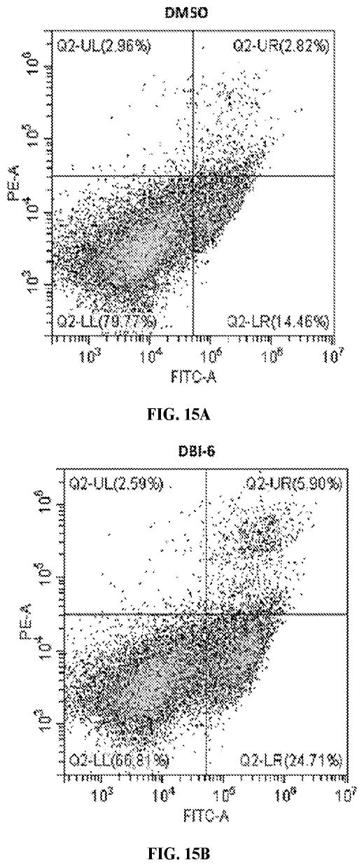

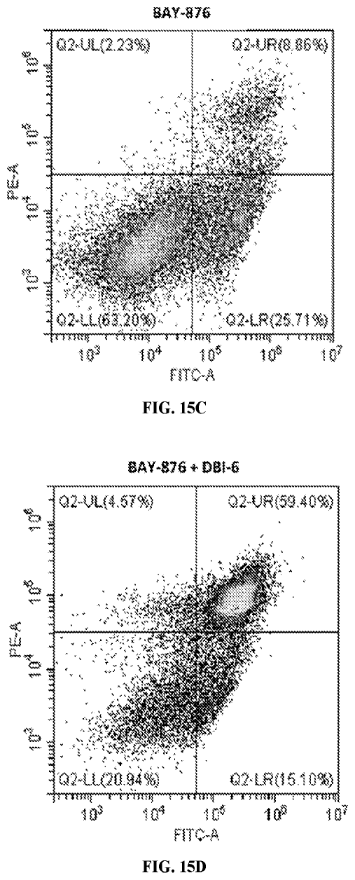

The combinational effects of DBI-6 and BAY-876 were also analyzed on CRC cell apoptosis. Annexin V-FITC was used to quantify the externalization of phosphatidylserine, while the dye propidium iodide (PI) was used to evaluate the loss of membrane integrity as a marker of apoptosis and necrosis, respectively (94). After 24 h treatment, the cells were measured by flow cytometry for Annexin V FITC and PI dual labeling. The apoptosis rate of the DMSO treated group was 17.28% (FIG. 15A). The percentage of apoptotic cells of DBI-6 (3 μM)-treated group was 30.61% and that of the BAY-876 (1 μM)-treated group was 34.57% (FIGS. 15B and 15C). The combination of DBI-6 with BAY-876 resulted in a significant increase in apoptosis rate of 74.5%. (FIG. 15D).

### Combinational Effects of DBI-6 and BAY-876 on Cell Signaling Pathways in CRC Cells.

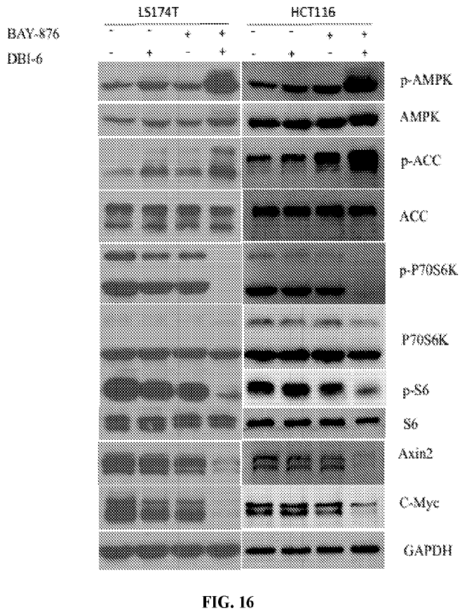

Three (3) μM of DBI-6 was demonstrated to activate AMPK and inhibited mTOR and Wnt signaling pathways in CRC cells (FIG. 11B). To test the combinational effects of DBI-1 and BAY-876, LS174T and HCT116 CRC cells were treated with 3 μM DBI-6, 1 μM BAY-876 or the combination of these two compounds. The combination of DBI-6 and BAY-876 was significanμy more effective than either DBI-6 or BAY-876 alone in activating AMPK and inhibiting mTOR and Wnt signaling pathways (FIG. 16).

### Analysis of Functional Enrichment of Differentially Expressed Genes

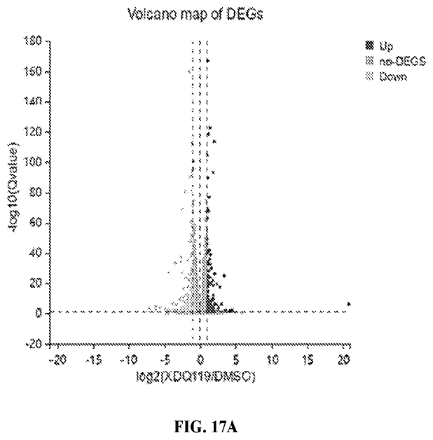

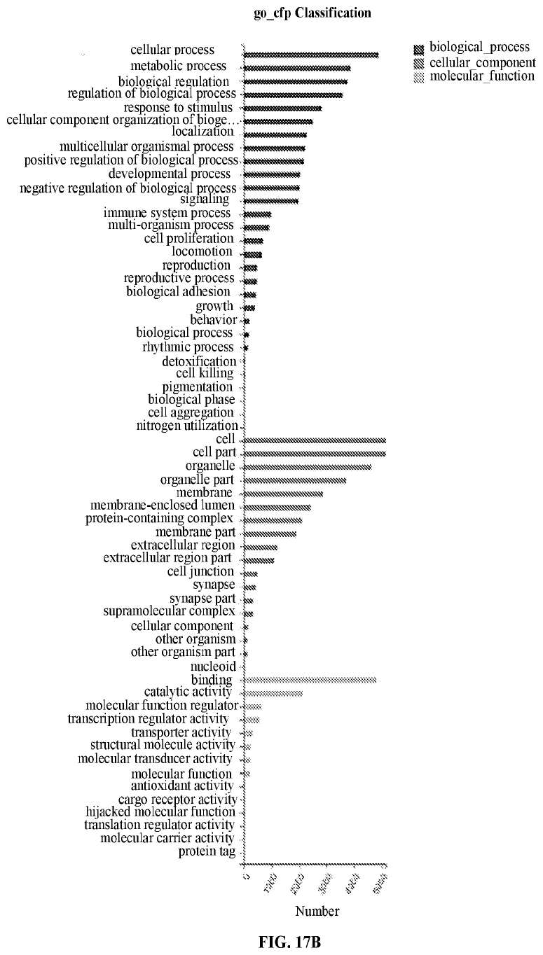

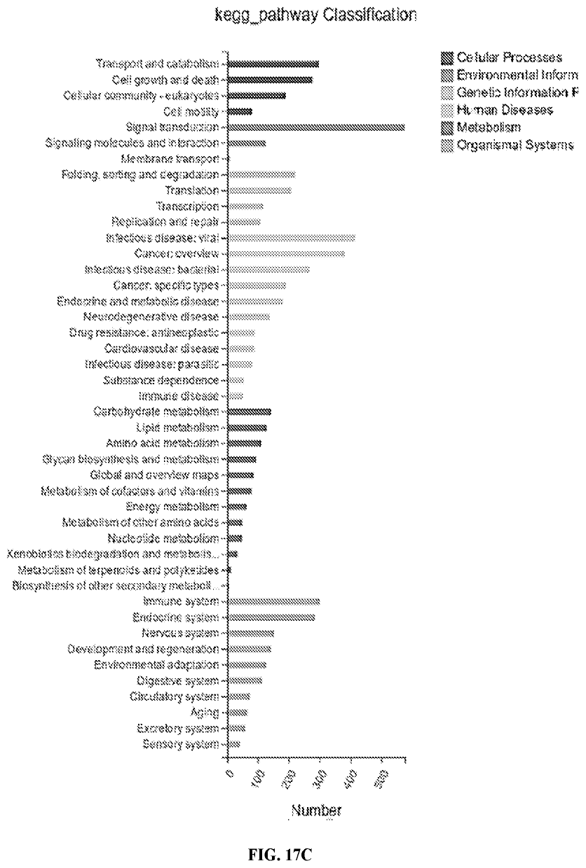

To further understand the biological effects of DBI-6, the transcriptomic changes in CRC cells were analyzed. LS174T cells were treated with DMSO or 5 μM DBI-6 for 24 h. In the DBI-6-treated cells, 2772 genes were down-regulated, and 2799 genes were up-regulated. Qvalue ≤0.05 and |log 2FC|≥1 (FIG. 17A). The Gene Ontology (GO) enrichment classification can visualize the distribution of the number of differential genes enriched in three components: biological process, cellular component, and molecular function (FIG. 17B). In biological process, cellular process, metabolic process, biological regulation, regulation of biological process and response to stimulus are the top 5 processes. Cell, cell part, organelle, organelle part and membrane are the most annotated cellular components. Binding and catalytic activity are the main molecular function. The classification of changed pathways mainly includes Cellular Processes, Environmental Information Processing, Genetic Information Processing, Human Diseases, Metabolism, and Organismal Systems (FIG. 17C). The GO analysis identified metabolic process as the second biological process. KEGG pathway analysis indicated signal transduction main pathways. These analyses were consistent with the above findings that DBI-6, a mitochondrial complex 1 inhibitor, inhibited mitochondrial energy metabolism and modulated Wnt, AMPK and mTOR signaling pathways.

### GLUT1 Inhibitor or Ketogenic Diet Enhanced the Therapeutic Efficacy of DBI-6 on CRC Xenograft Models.

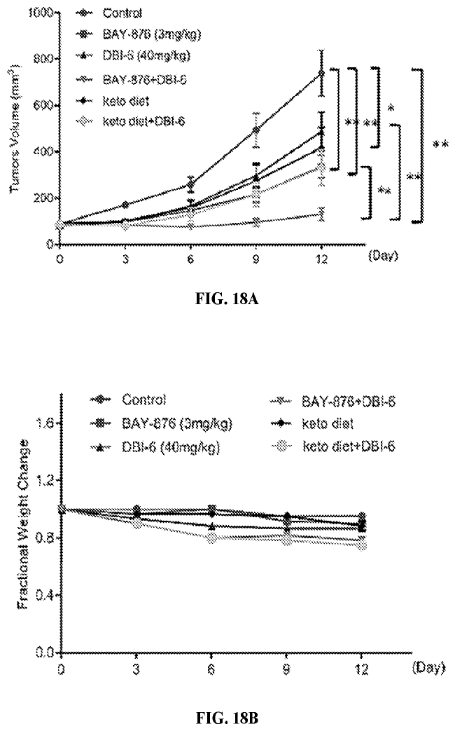

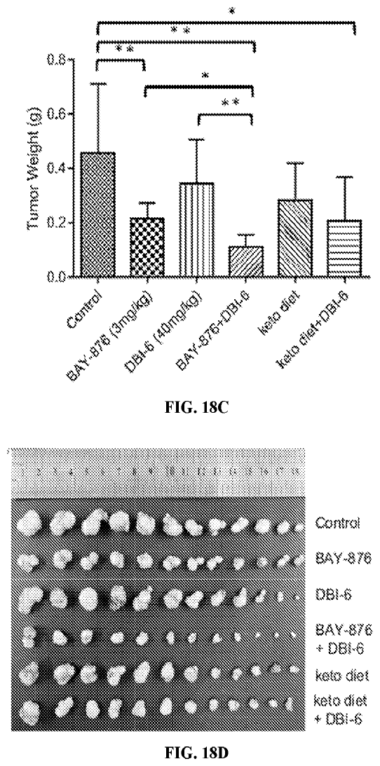

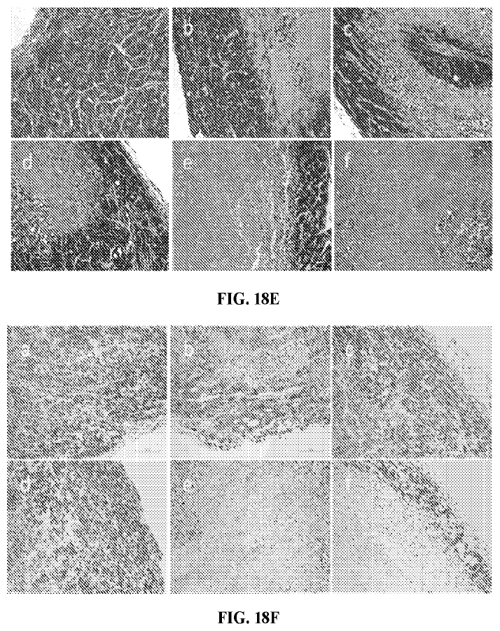

The in vivo therapeutic efficiencies of DBI-6 and its combination with GLUT1 inhibitor BAY-876 and ketogenic diet were explored in mouse models. A CRC xenograft model was established using LS174T cells in Rag1 mice. Tumor growth was inhibited by daily intraperitoneal (IP) administration of 40 mg/kg/day of DBI-6 or oral administration of 3 mg/kg of BAY-876 or daily ketogenic diet feeding for 12 days (FIG. 18A). The combination of BAY-876 at 3 mg/kg/day and DBI-6 at 40 mg/kg/day significanμy inhibited tumor growth than either compound alone (FIG. 18A). IP injection of DBI-6 at 40 mg/kg/day together with ketogenic diet feeding also significanμy inhibited tumor growth, but the effect was inferior to the combination of DBI-6 and BAY-876 (FIG. 18A). All the treatment groups showed no significant toxicity based on mice body weight growth rate (FIG. 18B). After 12 days of therapy, tumors were collected and weighed, and tumor slides were analyzed by H&E and Ki-67 staining (FIG. 18C-18F). Immunohistochemical results showed that the cell proliferation marker Ki-67 was significanμy reduced in tumor samples treated with the combination of DBI-6 and BAY-876 and DBI-6 in combination with ketogenic diet feeding (FIG. 18F). In conclusion, DBI-6 has a more prominent potential anticancer effect when used alone or in combination with other energy metabolism inhibitors, such as glucose transport inhibitors. The drug-diet combination also provides a therapeutic strategy for cancer treatment.

Combination of DBI-6 with BAY-876 Reduced Blood Glucose, Plasma TG, T-CHO and LDL-C.

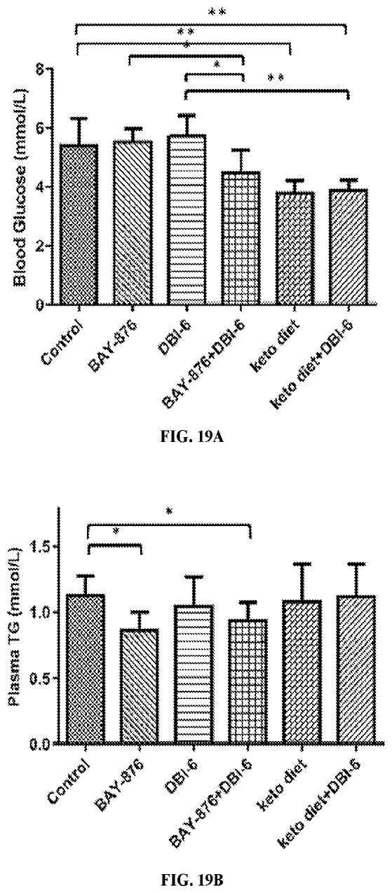

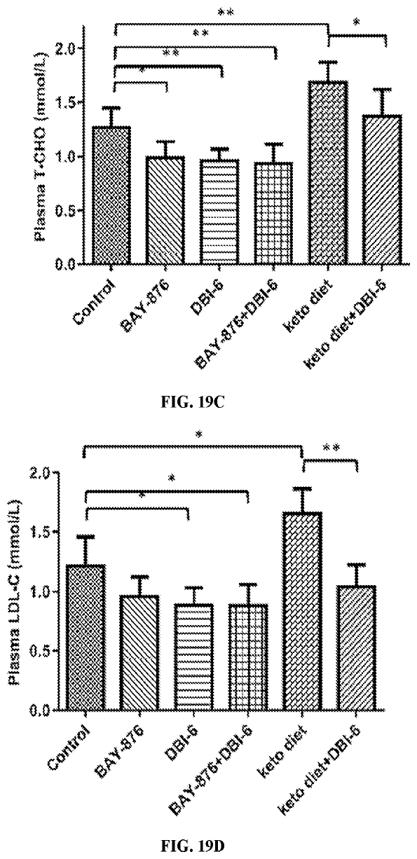

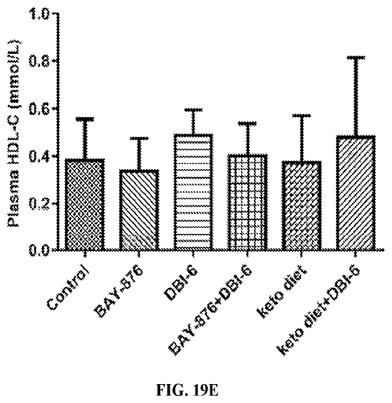

The DBI-6+BAY-876, ketogenic diet-fed and DBI-6+KD treatment group had a significant reduction in blood glucose (FIG. 19A). It has been shown that the synergistic effect of protein restriction and choline deficiency affects the combined metabolism and liver pathology in mice when nutritional fat content is high (95). Because high cholesterol levels are a concern with high-fat diets, analyses of triglycerides, cholesterol, LDL and HDL were performed (96). Consistent with these findings, mice fed the ketogenic diet showed a significant increase in cholesterol and LDL levels (FIGS. 19C and 19D). DBI-6 combined with BAY-876 significanμLy reduced triglyceride, cholesterol and LDL levels, which was better than ketogenic diet combined with DBI-6 (FIGS. 19B, 19C and 19D). There were no significant changes in HDL level among all groups. Taken together, these results indicated that DBI-6 in combination with BAY-876 is relatively healthy and nutritionally safe.

### Discussion

Diaminobutoxy-substituted isoflavonoid (DBIs) compounds were developed that inhibited the proliferation of CRC cells by targeting mitochondrial complex I. DBI-6 was identified as a leading DBI analog from SAR studies (FIG. 1). DBI-6 inhibited induced reprogramming of metabolic pathways in CRC cells. Combination of DBI-1 and BAY-876, a GLUT1 inhibitor, synergistically inhibited CRC cells in vitro and in vivo. In addition, ketogenic diet also enhanced the therapeutic efficacy of DBI-6 in CRC xenograft mouse models.

Mechanistically, DBI-6 inhibited mitochondrial complex I activity (FIG. 2), disrupted the OXPHOS and reduced ATP generation from mitochondria. Cancer cells must rely more on glycolysis for ATP production. BAY-876, as a GLUT1 inhibitor, reduced glucose uptake and inhibited glycolysis. Cancer cells must rely more on other energy sources, such as fatty acids and amino acids, which enter TCA and OXPHOS to produce ATP. Therefore, simultaneous inhibition of the two main energy supply pathways that are adopted by cancer cells can synergistically decrease the growth of cancer cells. Thus, this dual inhibition would result in a particularly effective inhibition of cancer cell growth and might overcome therapeutic resistance through metabolic plasticity (97, 98).

Other than metabolic inhibitors, the calorie-restricted ketogenic diet (KD) could also be used in cancer management. KD is a high-fat, low-carbohydrate, low-protein diet where extreme carbohydrate restriction mimics a fasted state, resulting in a decrease in blood glucose and the induction of ketone bodies (99, 100). Ketone bodies are suitable energy substitutes for normal cells with functional mitochondria, but have proven inappropriate for tumor cells, which have dysfunctional mitochondria and cannot compensate for glucose limitation by metabolizing ketone bodies (101-104). Decreased ketogenesis is a signature of CRC and KD can repress CRC growth (105).

In the study, the combination treatment of KD and DBI-6 significanμLy inhibited tumor growth, although the effect was weaker than that of the DBI-6/BAY-876 combination. The KD group and the DBI-6+KD treatment group showed a significant decrease in blood glucose (FIG. 19A), which is consistent with the results reported by Poff et al (106). The combination of DBI-6 and BAY-876 showed a significant decrease in triglycerides, cholesterol, and LDL. The DBI-6+KD treatment group was able to alleviate the high levels of cholesterol and LDL caused by KD feeding alone. These results indicate that the combination of DBI-6 and BAY-876 is nutritionally safer and more effective in inhibiting tumor growth. However, the therapeutic window of BAY-876 is very narrow, KD and other GLUT1 inhibitors should be further explored for combinational therapy.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. La Vecchia, S., and Sebastian, C. (2020) Metabolic pathways regulating colorectal cancer initiation and progression. Seminars in Cell & Developmental Biology 98, 63-70.
2. Neitzel, C., Demuth, P., Wittmann, S., and Fahrer, J. (2020) Targeting Altered Energy Metabolism in Colorectal Cancer: Oncogenic Reprogramming, the Central Role of the TCA Cycle and Therapeutic Opportunities. Cancers 12.
3. Samec, M., Liskova, A., Koklesova, L., Samuel, S. M., Zhai, K., Buhrmann, C., Varghese, E., Abotaleb, M., Qaradakhi, T., Zulli, A., Kello, M., Mojzis, J., Zubor, P., Kwon, T. K., Shakibaei, M., Busselberg, D., Sarria, G. R., Golubnitschaja, O., and Kubatka, P. (2020) Flavonoids against the Warburg phenotype—concepts of predictive, preventive and personalised medicine to cut the Gordian knot of cancer cell metabolism. EPMA Journal 11, 377-398.
4. Mukha, A., and Dubrovska, A. (2020) Metabolic Targeting of Cancer Stem Cells. Frontiers in Oncology 10.
5. Ward, P. S., and Thompson, C. B. (2012) Metabolic reprogramming: a cancer hallmark even warburg did not anticipate. Cancer Cell 21, 297-308.
6. Cairns, R. A., Harris, I., McCracken, S., and Mak, T. W. (2011) Cancer cell metabolism. Cold Spring Harb Symp Quant Biol 76, 299-311.
7. Pavlova, N. N., and Thompson, C. B. (2016) The Emerging Hallmarks of Cancer Metabolism. Cell Metab 23, 27-47.
8. Warburg, O. (1924) Über den Stoffwechsel der Carcinomzelle. Naturwissenschaften 12, 1131-1137.
9. Warburg, O., Negelein, E., and Posener, K. (1924) Versuche an Überlebendem Carcinomgewebe. Klinische Wochenschrft 3, 1062-1064.
10. Vander Heiden, M. G., Cantley, L. C., and Thompson, C. B. (2009) Understanding the Warburg effect: the metabolic requirements of cell proliferation. Science (New York, N.Y.) 324, 10291033.
11. Li, C., Zhang, G., Zhao, L., Ma, Z., and Chen, H. (2016) Metabolic reprogramming in cancer cells: glycolysis, glutaminolysis, and Bel-2 proteins as novel therapeutic targets for cancer. World journal of surgical oncology 14, 15.
12. Deng, G., Shen, J., Yin, M., McManus, J., Mathieu, M., Gee, P., He, T., Shi, C., Bedel, O., McLean, L. R., Le-Strat, F., Zhang, Y., Marquette, J. P., Gao, Q., Zhang, B., Rak, A., Hoffmann, D., Rooney, E., Vassort, A., Englaro, W., Li, Y., Patel, V., Adrian, F., Gross, S., Wiederschain, D., Cheng, H., and Licht, S. (2015) Selective inhibition of mutant isocitrate dehydrogenase 1 (IDH1) via disruption of a metal binding network by an allosteric small molecule. The Journal of biological chemistry 290, 762-774.
13. Cho, H., Lee, Y. S., Kim, J., Chung, J. Y., and Kim, J. H. (2013) Overexpression of glucose transporter-1 (GLUT-1) predicts poor prognosis in epithelial ovarian cancer. Cancer investigation 31, 607-615.
14. Ganapathy-Kanniappan, S., and Geschwind, J. F. (2013) Tumor glycolysis as a target for cancer therapy: progress and prospects. Molecular cancer 12, 152.
15. Macheda, M. L., Rogers, S., and Best, J. D. (2005) Molecular and cellular regulation of glucose transporter (GLUT) proteins in cancer. Journal of cellular physiology 202, 654-662.
16. Vardhana, P. A., and Illsley, N. P. (2002) Transepithelial glucose transport and metabolism in BeWo choriocarcinoma cells. Placenta 23, 653-660.
17. Ancey, P. B., Contat, C., and Meylan, E. (2018) Glucose transporters in cancer—from tumor cells to the tumor microenvironment. The FEBS journal 285, 2926-2943.
18. Carvalho, K. C., Cunha, I. W., Rocha, R. M., Ayala, F. R., Cajaiba, M. M., Begnami, M. D., Vilela, R. S., Paiva, G. R., Andrade, R. G., and Soares, F. A. (2011) GLUT1 expression in malignant tumors and its use as an immunodiagnostic marker. Clinics (Sao Paulo, Brazil) 66, 965-972.
19. Chan, D. A., Sutphin, P. D., Nguyen, P., Turcotte, S., Lai, E. W., Banh, A., Reynolds, G. E., Chi, J. T., Wu, J., Solow-Cordero, D. E., Bonnet, M., Flanagan, J. U., Bouley, D. M., Graves, E. E., Denny, W. A., Hay, M. P., and Giaccia, A. J. (2011) Targeting GLUT1 and the Warburg effect in renal cell carcinoma by chemical synthetic lethality. Science translational medicine 3, 94ra70.
20. Wu, Q., Ba-Alawi, W., Deblois, G., Cruickshank, J., Duan, S., Lima-Fernandes, E., Haight, J., Tonekaboni, S. A. M., Fortier, A. M., Kuasne, H., McKee, T. D., Mahmoud, H., Kushida, M., Cameron, S., Dogan-Artun, N., Chen, W., Nie, Y., Zhang, L. X., Vellanki, R. N., Zhou, S., Prinos, P., Wouters, B. G., Dirks, P. B., Done, S. J., Park, M., Cescon, D. W., Haibe-Kains, B., Lupien, M., and Arrowsmith, C. H. (2020) GLUT1 inhibition blocks growth of RB1-positive triple negative breast cancer. Nature communications 11, 4205.
21. Liu, Y., Cao, Y., Zhang, W., Bergmeier, S., Qian, Y., Akbar, H., Colvin, R., Ding, J., Tong, L., Wu, S., Hines, J., and Chen, X. (2012) A small-molecule inhibitor of glucose transporter 1 downregulates glycolysis, induces cell-cycle arrest, and inhibits cancer cell growth in vitro and in vivo. Molecular cancer therapeutics 11, 1672-1682.
22. Siebeneicher, H., Cleve, A., Rehwinkel, H., Neuhaus, R., Heisler, I., Müller, T., Bauser, M., and Buchmann, B. (2016) Identification and Optimization of the First Highly Selective GLUT1 Inhibitor BAY-876. ChemMedChem 11, 2261-2271.
23. Ma, Y., Wang, W., Idowu, M. O., Oh, U., Wang, X. Y., Temkin, S. M., and Fang, X. (2018) Ovarian Cancer Relies on Glucose Transporter 1 to Fuel Glycolysis and Growth: Anti-Tumor Activity of BAY-876. Cancers (Basel) 11.
24. Reckzeh, E. S., Karageorgis, G., Schwalfenberg, M., Ceballos, J., Nowacki, J., Stroet, M. C. M., Binici, A., Knauer, L., Brand, S., Choidas, A., Strohmann, C., Ziegler, S., and Waldmann, H. (2019) Inhibition of Glucose Transporters and Glutaminase Synergistically Impairs Tumor Cell Growth. Cell Chem Biol 26, 1214-1228 e1225.
25. Weinberg, S. E., and Chandel, N. S. (2015) Targeting mitochondria metabolism for cancer therapy. Nat Chem Biol 11, 9-15.
26. Chandel, N. S. (2014) Mitochondria as signaling organelles. BMC Biol 12, 34.
27. DeBerardinis, R. J., and Chandel, N. S. (2016) Fundamentals of cancer metabolism. Sci Adv 2, e1600200.
28. Porporato, P. E., Filigheddu, N., Pedro, J. M. B.-S., Kroemer, G., and Galluzzi, L. (2018) Mitochondrial metabolism and cancer. Cell Research 28, 265-280.
29. Ashton, T. M., McKenna, W. G., Kunz-Schughart, L. A., and Higgins, G. S. (2018) Oxidative Phosphorylation as an Emerging Target in Cancer Therapy. Clinical cancer research: an official journal of the American Association for Cancer Research 24, 2482-2490.
30. Santidrian, A. F., Matsuno-Yagi, A., Ritland, M., Seo, B. B., LeBoeuf, S. E., Gay, L. J., Yagi, T., and Felding-Habermann, B. (2013) Mitochondrial complex I activity and NAD+/NADH balance regulate breast cancer progression. The Journal of clinical investigation 123, 1068-1081.
31. Palmer, G., Horgan, D. J., Tisdale, H., Singer, T. P., and Beinert, H. (1968) Studies on the respiratory chain-linked reduced nicotinamide adenine dinucleotide dehydrogenase. XIV. Location of the sites of inhibition of rotenone, barbiturates, and piericidin by means of electron paramagnetic resonance spectroscopy. The Journal of biological chemistry 243, 844-847.
32. Heinz, S., Freyberger, A., Lawrenz, B., Schladt, L., Schmuck, G., and Ellinger-Ziegelbauer, H. (2017) Mechanistic Investigations of the Mitochondrial Complex I Inhibitor Rotenone in the Context of Pharmacological and Safety Evaluation. Scientific reports 7, 45465.
33. Hirsch, H. A., Iliopoulos, D., Tsichlis, P. N., and Struhl, K. (2009) Metformin selectively targets cancer stem cells, and acts together with chemotherapy to block tumor growth and prolong remission. Cancer Res 69, 7507-7511.
34. Suissa, S., and Azoulay, L. (2014) Metformin and cancer: mounting evidence against an association. Diabetes Care 37, 1786-1788.
35. Evans, J. M., Donnelly, L. A., Emslie-Smith, A. M., Alessi, D. R., and Morris, A. D. (2005) Metformin and reduced risk of cancer in diabetic patients. BMJ 330, 1304-1305.
36. Kasznicki, J., Sliwinska, A., and Drzewoski, J. (2014) Metformin in cancer prevention and therapy. Ann Transl Med 2, 57.
37. Chen, J. (2011) Multiple signal pathways in obesity-associated cancer. Obes Rev 12, 1063-1070.
38. Jafari, N., Drury, J., Morris, A. J., Onono, F. O., Stevens, P. D., Gao, T., Liu, J., Wang, C., Lee, E. Y., Weiss, H. L., Evers, B. M., and Zaytseva, Y. Y. (2019) De Novo Fatty Acid Synthesis-Driven Sphingolipid Metabolism Promotes Metastatic Potential of Colorectal Cancer. Mol Cancer Res 17, 140-152.
39. Sinner, H. F., Johnson, J., Rychahou, P. G., Watt, D. S., Zaytseva, Y. Y., Liu, C., and Evers, B. M. (2019) Novel chemotherapeutic agent, FND-4b, activates AMPK and inhibits colorectal cancer cell proliferation. PLoS One 14, e0224253.
40. Wen, Y. A., Xing, X., Harris, J. W., Zaytseva, Y. Y., Mitov, M. I., Napier, D. L., Weiss, H. L., Mark Evers, B., and Gao, T. (2017) Adipocytes activate mitochondrial fatty acid oxidation and autophagy to promote tumor growth in colon cancer. Cell Death Dis 8, e2593.
41. Zhang, W., Sviripa, V. M., Xie, Y., Yu, T., Haney, M. G., Blackburn, J. S., Adeniran, C. A., Zhan, C. G., Watt, D. S., and Liu, C. (2020) Epigenetic Regulation of Wnt Signaling by Carboxamide-Substituted Benzhydryl Amines that Function as Histone Demethylase Inhibitors. iScience 23, 101795
42. Balasubramanian S, N. MG. (2000) An efficient “one-pot” synthesis of isoflavones. Synth Commun 30, 469-484.
43. Sum T J S, T. H.; Galloway WRJD; Twigg D G; Ciardiello J J; Spring D R. (2018) Synthesis of structurally diverse biflavonoids. Tetrahedron 74, 5089-5101.
44. Gao G Y, Li D J, and Keung W M (2003) Synthesis of daidzin analogues as potential agents for alcohol abuse. Bioorg Med Chem 11, 4069-4081 [PubMed: 12927869]
45. Barker N, Hurlstone A, Musisi H, Miles A, Bienz M, and Clevers H (2001) The chromatin remodelling factor Brg-1 interacts with beta-catenin to promote target gene activation. EMBO J 20, 4935-4943 [PubMed: 11532957]
46. Jafari N, Drury J, Morris A J, Onono F O, Stevens P D, Gao T, Liu J, Wang C, Lee E Y, Weiss H L, Evers B M, and Zaytseva Y Y (2019) De Novo Fatty Acid Synthesis-Driven Sphingolipid Metabolism Promotes Metastatic Potential of Colorectal Cancer. Mol Cancer Res 17, 140-152 [PubMed: 30154249]
47. Sinner H F, Johnson J, Rychahou P G, Watt D S, Zaytseva Y Y, Liu C, and Evers B M (2019) Novel chemotherapeutic agent, FND-4b, activates AMPK and inhibits colorectal cancer cell proliferation. PLoS One 14, e0224253 [PubMed: 31648230]
48. Xie Y, Kril L M, Yu T, Zhang W, Frasinyuk M S, Bondarenko S P, Kondratyuk K M, Hausman E, Martin Z M, Wyrebek P P, Liu X, Deaciuc A, Dwoskin L P, Chen J, Zhu H, Zhan C-G, Sviripa V M, Blackburn J, Watt D S, and Liu C (2019) Semisynthetic aurones inhibit tubulin polymerization at the colchicine-binding site and repress PC-3 tumor xenografts in nude mice and myc-induced T-ALL in zebrafish. Scientific Reports 9, 6439 [PubMed: 31015569]
49. Ianevski A, Giri A K, and Aittokallio T (2020) SynergyFinder 2.0: visual analytics of multi-drug combination synergies. Nucleic Acids Research 48, W488-W493 [PubMed: 32246720]
50. Zhang W, Sviripa V M, Kril L M, Yu T, Xie Y, Hubbard W B, Sullivan P G, Chen X, Zhan C G, Yang-Hartwich Y, Evers B M, Spear B T, Gedaly R, Watt D S, and Liu C (2019) An Underlying Mechanism of Dual Wnt Inhibition and AMPK Activation: Mitochondrial Uncouplers Masquerading as Wnt Inhibitors. J Med Chem 62, 11348-11358 [PubMed: 31774672]
51. Wen Y A, Xing X, Harris J W, Zaytseva Y Y, Mitov M I, Napier D L, Weiss H L, Mark Evers B, and Gao T (2017) Adipocytes activate mitochondrial fatty acid oxidation and autophagy to promote tumor growth in colon cancer. Cell death & disease 8, e2593 [PubMed: 28151470]
52. Tang Z, Li C, Kang B, Gao G, Li C, and Zhang Z (2017) GEPIA: a web server for cancer and normal gene expression profiling and interactive analyses. Nucleic Acids Res 45, W98-W102 [PubMed: 28407145]
53. Frasinyuk M S, Mrug G P, Bondarenko S P, Khilya V P, Sviripa V M, Syrotchuk O A, Zhang W, Cai X, Fiandalo M V, Mohler J L, Liu C, and Watt D S (2016) Antineoplastic Isoflavonoids Derived from Intermediate ortho-Quinone Methides Generated from Mannich Bases. ChemMedChem 11, 600-611 [PubMed: 26889756
54. Frasinyuk M S, Mrug G P, Bondarenko S P, Sviripa V M, Zhang W, Cai X, Fiandalo M V, Mohler J L, Liu C, and Watt D S (2015) Application of Mannich bases to the synthesis of hydroxymethylated isoflavonoids as potential antineoplastic agents. Org Biomol Chem 13, 11292-11301 [PubMed: 26416505]
55. Chung S T, Huang Y T, Hsiung H Y, Huang W H, Yao C W, and Lee A R (2015) Novel daidzein analogs and their in vitro anti-influenza activities. Chem Biodivers 12, 685-696 [PubMed: 25879510]
56. Miao J, Cui H, Jin J, Lai F, Wen H, Zhang X, Ruda G F, Chen X, and Yin D (2015) Development of 3-alkyl-6-methoxy-7-hydroxy-chromones (AMHCs) from natural isoflavones, a new class of fluorescent scaffolds for biological imaging. Chem Commun (Camb) 51, 881-884 [PubMed: 25429667]
57. Xie Y, Zhang W, Guo L, Kril L M, Begley K L, Sviripa V M, Chen X, Liu X, Lee E Y, He D, Wang C, Gao T, Liu X, Evers B M, Watt D S, and Liu C (2021) Potent Synergistic Effect on C-Myc-Driven Colorectal Cancers Using a Novel Indole-Substituted Quinoline with a Plk1 Inhibitor. Molecular cancer therapeutics 20, 1893-1903 [PubMed: 34376582]
58. Zhang W, Sviripa V M, Xie Y, Yu T, Haney M G, Blackburn J S, Adeniran C A, Zhan C G, Watt D S, and Liu C (2020) Epigenetic Regulation of Wnt Signaling by Carboxamide-Substituted Benzhydryl Amines that Function as Histone Demethylase Inhibitors. iScience 23, 101795 [PubMed: 33305174]
59. Burikhanov R, Sviripa V M, Hebbar N, Zhang W, Layton W J, Hamza A, Zhan C G, Watt D S, Liu C, and Rangnekar V M (2014) Arylquins target vimentin to trigger Par-4 secretion for tumor cell apoptosis. Nat Chem Biol 10, 924-926 [PubMed: 25218743]
60. Frasinyuk M S, Zhang W, Wyrebek P, Yu T, Xu X, Sviripa V M, Bondarenko S P, Xie Y, Ngo H X, Morris A J, Mohler J L, Fiandalo M V, Watt D S, and Liu C (2017) Developing antineoplastic agents that target peroxisomal enzymes: cytisine-linked isoflavonoids as inhibitors of hydroxysteroid 17-beta-dehydrogenase-4 (HSD17B4). Org Biomol Chem 15, 7623-7629 [PubMed: 28868548]
61. Zhang W, Sviripa V, Chen X, Shi J, Yu T, Hamza A, Ward N D, Kril L M, Vander Kooi C W, Zhan C G, Evers B M, Watt D S, and Liu C (2013) Fluorinated N,N-dialkylaminostilbenes repress colon cancer by targeting methionine S-adenosyltransferase 2A. ACS Chem Biol 8, 796-803 [PubMed: 23363077]
62. Zhang W, Sviripa V, Kril L M, Chen X, Yu T, Shi J, Rychahou P, Evers B M, Watt D S, and Liu C (2011) Fluorinated N,N-dialkylaminostilbenes for Wnt pathway inhibition and colon cancer repression. J Med Chem 54, 1288-1297 [PubMed: 21291235]
63. Kril L M, Vilchez V, Jiang J, Turcios L, Chen C, Sviripa V M, Zhang W, Liu C, Spear B, Watt D S, and Gedaly R (2015) N-Aryl benzenesulfonamide inhibitors of [3H]-thymidine incorporation and β-catenin signaling in human hepatocyte-derived Huh-7 carcinoma cells. Bioorganic & medicinal chemistry letters 25, 3897-3899 [PubMed: 26243371]
64. Zhang W, Sviripa V M, Xie Y, Yu T, Haney M G, Blackburn J S, Adeniran C A, Zhan C-G, Watt D S, and Liu C (2020) Epigenetic Regulation of Wnt Signaling by Carboxamide-Substituted Benzhydryl Amines that Function as Histone Demethylase Inhibitors. iScience
65. Barron C C, Bilan P J, Tsakiridis T, and Tsiani E (2016) Facilitative glucose transporters: Implications for cancer detection, prognosis and treatment. Metabolism: clinical and experimental 65, 124-139 [PubMed: 26773935]
66. Shi Y, Liu S, Ahmad S, and Gao Q (2018) Targeting Key Transporters in Tumor Glycolysis as a Novel Anticancer Strategy. Curr Top Med Chem 18, 454-466 [PubMed: 29788889]
67. Zhang W, Liu Y, Chen X, and Bergmeier S C (2010) Novel inhibitors of basal glucose transport as potential anticancer agents. Bioorganic & medicinal chemistry letters 20, 2191-2194 [PubMed:20194024]
68. Kang S A, O'Neill D J, Machl A W, Lumpkin C J, Galda S N, Sengupta S, Mahoney S J, Howell J J, Molz L, Hahm S, Vlasuk G P, and Saiah E (2019) Discovery of Small-Molecule Selective mTORC1 Inhibitors via Direct Inhibition of Glucose Transporters. Cell Chem Biol 26, 1203-1213.e1213 [PubMed: 31231029]
69. Di Dedda C, Vignali D, Piemonti L, and Monti P (2019) Pharmacological Targeting of GLUT1 to Control Autoreactive T Cell Responses. Int J Mol Sci 20
70. Wu Q, Heidenreich D, Zhou S, Ackloo S, Kramer A, Nakka K, Lima-Fernandes E, Deblois G, Duan S, Vellanki R N, Li F, Vedadi M, Dilworth J, Lupien M, Brennan P E, Arrowsmith C H, Müller S, Fedorov O, Filippakopoulos P, and Knapp S (2019) A chemical toolbox for the study of bromodomains and epigenetic signaling. Nat Commun 10, 1915 [PubMed: 31015424]
71. Sun L, Yin Y, Clark L H, Sun W, Sullivan S A, Tran A Q, Han J, Zhang L, Guo H, Madugu E, Pan T, Jackson A L, Kilgore J, Jones H M, Gilliam T P, Zhou C, and Bae-Jump V L (2017) Dual inhibition of glycolysis and glutaminolysis as a therapeutic strategy in the treatment of ovarian cancer. Oncotarget 8, 63551-63561 [PubMed: 28969010]
72. Camarda R, Zhou A Y, Kohnz R A, Balakrishnan S, Mahieu C, Anderton B, Eyob H, Kajimura S, Tward A, Krings G, Nomura D K, and Goga A (2016) Inhibition of fatty acid oxidation as a therapy for MYC-overexpressing triple-negative breast cancer. Nat Med 22, 427-432 [PubMed: 26950360]
73. Reis L M D, Adamoski D, Ornitz Oliveira Souza R, Rodrigues Ascenção CF, Sousa de Oliveira K R, Corrêa-da-Silva F, Malta de Si Patroni, F., Meira Dias, M., Consonni, S. R., Mendes de Moraes-Vieira, P. M., Silber, A. M., and Dias, S. M. G. (2019) Dual inhibition of glutaminase and carnitine palmitoyltransferase decreases growth and migration of glutaminase inhibition-resistant triple-negative breast cancer cells. The Journal of biological chemistry 294, 9342-9357 [PubMed: 31040181]
74. Naguib A, Mathew G, Reczek C R, Watrud K, Ambrico A, Herzka T, Salas I C, Lee M F, El-Amine N, Zheng W, Di Francesco M E, Marszalek J R, Pappin D J, Chandel N S, and Trotman L C (2018) Mitochondrial Complex I Inhibitors Expose a Vulnerability for Selective Killing of Pten-Null Cells. Cell Rep 23, 58-67 [PubMed: 29617673]
75. Garcia-Bermudez J, Baudrier L, La K, Zhu X G, Fidelin J, Sviderskiy V O, Papagiannakopoulos T, Molina H, Snuderl M, Lewis C A, Possemato R L, and Birsoy K (2018) Aspartate is a limiting metabolite for cancer cell proliferation under hypoxia and in tumours. Nat Cell Biol 20, 775-781 [PubMed: 29941933]
76. Molina J R, Sun Y, Protopopova M, Gera S, Bandi M, Bristow C, McAfoos T, Morlacchi P, Ackroyd J, Agip A A, Al-Atrash G, Asara J, Bardenhagen J, Carrillo C C, Carroll C, Chang E, Ciurea S, Cross J B, Czako B, Deem A, Daver N, de Groot J F, Dong J W, Feng N, Gao G, Gay J, Do M G, Greer J, Giuliani V, Han J, Han L, Henry V K, Hirst J, Huang S, Jiang Y, Kang Z, Khor T, Konoplev S, Lin Y H, Liu G, Lodi A, Lofton T, Ma H, Mahendra M, Matre P, Mullinax R, Peoples M, Petrocchi A, Rodriguez-Canale J, Serreli R, Shi T, Smith M, Tabe Y, Theroff J, Tiziani S, Xu Q, Zhang Q, Muller F, DePinho R A, Toniatti C, Draetta G F, Heffernan T P, Konopleva M, Jones P, Di Francesco M E, and Marszalek J R (2018) An inhibitor of oxidative phosphorylation exploits cancer vulnerability. Nat Med 24, 1036-1046 [PubMed: 29892070]
77. Sullivan L B, Gui D Y, Hosios A M, Bush L N, Freinkman E, and Vander Heiden M G (2015) Supporting Aspartate Biosynthesis Is an Essential Function of Respiration in Proliferating Cells. Cell 162, 552-563 [PubMed: 26232225]
78. Birsoy K, Wang T, Chen W W, Freinkman E, Abu-Remaileh M, and Sabatini D M (2015) An Essential Role of the Mitochondrial Electron Transport Chain in Cell Proliferation Is to Enable Aspartate Synthesis. Cell 162, 540-551 [PubMed: 26232224]
79. Feron O (2019) The many metabolic sources of acetyl-CoA to support histone acetylation and influence cancer progression. Ann Transl Med 7, S277 [PubMed: 32015996]
80. Kernytsky A, Wang F, Hansen E, Schalm S, Straley K, Gliser C, Yang H, Travins J, Murray S, Dorsch M, Agresta S, Schenkein D P, Biller S A, Su S M, Liu W, and Yen K E (2015) IDH2 mutation-induced histone and DNA hypermethylation is progressively reversed by small-molecule inhibition. Blood 125, 296-303 [PubMed: 25398940]
81. Murai M, and Miyoshi H (2016) Current topics on inhibitors of respiratory complex I. Biochim Biophys Acta 1857, 884-891 [PubMed: 26625959]
82. LaMoia T E, and Shulman G I (2021) Cellular and Molecular Mechanisms of Metformin Action. Endocr Rev 42, 77-96 [PubMed: 32897388]
83. Biller, L. H., and Schrag, D. (2021) Diagnosis and Treatment of Metastatic Colorectal Cancer: A Review. JAMA 325, 669-685
84. Bray, F., Ferlay, J., Soerjomataram, I., Siegel, R. L., Torre, L. A., and Jemal, A. (2018) Global cancer statistics 2018: GLOBOCAN estimates of incidence and mortality worldwide for 36 cancers in 185 countries. CA Cancer J Clin 68, 394-424
85. Hanahan, D., and Weinberg, R. A. (2011) Hallmarks of cancer: the next generation. Cell 144, 646-674
86. Warburg, O. H. (1930) The metabolism of tumours: investigations from the Kaiser Wilhelm Institute for Biology, Berlin-Dahlem, Constable & Company Limited
87. Warburg, O. J. S. (1956) On the origin of cancer cells. 123, 309-314
88. Warburg, O. J. S. (1956) On respiratory impairment in cancer cells. 124, 269-270
89. Nolfi-Donegan, D., Braganza, A., and Shiva, S. (2020) Mitochondrial electron transport chain: Oxidative phosphorylation, oxidant production, and methods of measurement. Redox Biology 37, 101674
90. Guo, L., Zhang, W., Xie, Y., Chen, X., Olmstead, E. E., Lian, M., Zhang, B., Zaytseva, Y. Y., Evers, B. M., Spielmann, H. P., Liu, X., Watt, D. S., and Liu, C. (2022) Diaminobutoxy-substituted Isoflavonoid (DBI-1) Enhances the Therapeutic Efficacy of GLUT1 Inhibitor BAY-876 by Modulating Metabolic Pathways in Colon Cancer Cells. Molecular cancer therapeutics 21, 740-750
91. Ramachandran, R., Heuck, A. P., Tweten, R. K., and Johnson, A. E. (2002) Structural insights into the membrane-anchoring mechanism of a cholesterol-dependent cytolysin. Nature structural biology 9, 823-827
92. Heuck, A. P., Moe, P. C., and Johnson, B. B. (2010) The cholesterol-dependent cytolysin family of gram-positive bacterial toxins. Sub-cellular biochemistry 51, 551-577
93. Sanyal, S., Claessen, J. H., and Ploegh, H. L. (2012) A viral deubiquitylating enzyme restores dislocation of substrates from the endoplasmic reticulum (ER) in semi-intact cells. The Journal of biological chemistry 287, 23594-23603
94. Rodrigues, A., Bomfim, L. M., Neves, S. P., Soares, M. B. P., Dias, R. B., Valverde, L. F., Rocha, C. A. G., Costa, E. V., da Silva, F. M. A., Rocha, W. C., Koolen, H. H. F., and Bezerra, D. P. (2021) Tingenone and 22-hydroxytingenone target oxidative stress through downregulation of thioredoxin, leading to DNA double-strand break and JNK/p38-mediated apoptosis in acute myeloid leukemia HL-60 cells. Biomed Pharmacother 142, 112034
95. Schugar, R. C., Huang, X., Moll, A. R., Brunt, E. M., and Crawford, P. A. (2013) Role of choline deficiency in the Fatty liver phenotype of mice fed a low protein, very low carbohydrate ketogenic diet. PLoS One 8, e74806
96. Kwiterovich, J. P. O., Vining, E. P. G., Pyzik, P., Skolasky, J. R., and Freeman, J. M. (2003) Effect of a High-Fat Ketogenic Diet on Plasma Levels of Lipids, Lipoproteins, and Apolipoproteins in Children. JAMA 290, 912-920
97. Marin-Hernandez, A., López-Ramirez, S. Y., Del Mazo-Monsalvo, I., Gallardo-Pérez, J. C., Rodríguez-Enríquez, S., Moreno-Sánchez, R., and Saavedra, E. (2014) Modeling cancer glycolysis under hypoglycemia, and the role played by the differential expression of glycolytic isoforms. The FEBS journal 281, 3325-3345
98. Nagamatsu, S., Sawa, H., Inoue, N., Nakamichi, Y., Takeshima, H., and Hoshino, T. (1994) Gene expression of GLUT3 glucose transporter regulated by glucose in vivo in mouse brain and in vitro in neuronal cell cultures from rat embryos. The Biochemical journal 300 (Pt 1), 125-131
99. Zhou, W., Mukherjee, P., Kiebish, M. A., Markis, W. T., Mantis, J. G., and Seyfried, T. N. (2007) The calorically restricted ketogenic diet, an effective alternative therapy for malignant brain cancer. Nutrition & metabolism 4, 5
100. VanItallie, T. B., and Nufert, T. H. (2003) Ketones: metabolism's ugly duckling. Nutrition reviews 61, 327-341
101. Veech, R. L. (2004) The therapeutic implications of ketone bodies: the effects of ketone bodies in pathological conditions: ketosis, ketogenic diet, redox states, insulin resistance, and mitochondrial metabolism. Prostaglandins, leukotrienes, and essential fatty acids 70, 309-319
102. Maurer, G. D., Brucker, D. P., Bahr, O., Harter, P. N., Hattingen, E., Walenta, S., Mueller-Klieser, W., Steinbach, J. P., and Rieger, J. (2011) Differential utilization of ketone bodies by neurons and glioma cell lines: a rationale for ketogenic diet as experimental glioma therapy. BMC cancer 11, 315
103. Chang, H. T., Olson, L. K., and Schwartz, K. A. (2013) Ketolytic and glycolytic enzymatic expression profiles in malignant gliomas: implication for ketogenic diet therapy. Nutrition & metabolism 10, 47
104. Boland, M. L., Chourasia, A. H., and Macleod, K. F. (2013) Mitochondrial dysfunction in cancer. Frontiers in oncology 3, 292
105. Wei, R., Zhou, Y., Li, C., Rychahou, P., Zhang, S., Titlow, W. B., Bauman, G., Wu, Y., Liu, J., Wang, C., Weiss, H. L., Evers, B. M., and Wang, Q. (2022) Ketogenesis Attenuates KLF5-Dependent Production of CXCL12 to Overcome the Immunosuppressive Tumor Microenvironment in Colorectal Cancer. Cancer Res 82, 1575-1588
106. Poff, A. M., Ari, C., Seyfried, T. N., and D'Agostino, D. P. (2013) The ketogenic diet and hyperbaric oxygen therapy prolong survival in mice with systemic metastatic cancer. PloS one 8, e65522

It will be understood that various details of the presently disclosed subject matter can be changed without departing from the scope of the subject matter disclosed herein.

Furthermore, the foregoing description is for the purpose of illustration only, and not for the purpose of limitation.

What is claimed is:

1. A compound having a structure according to Formula I, and salts thereof: 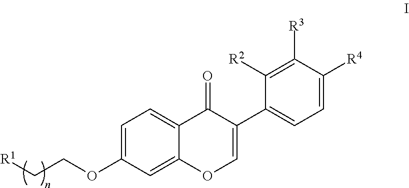
wherein R¹ is heterocycloalkyl or secondary aminoalkyl;
wherein each of R², R³, and R⁴ is independently selected from the group consisting of H, halo, and alkoxy; and
wherein n is from 1 to 10.

2. The compound of claim 1, wherein R¹ comprises a six-membered heterocycloalkyl.

3. The compound of claim 2, wherein the compound has a structure according to Formula II: 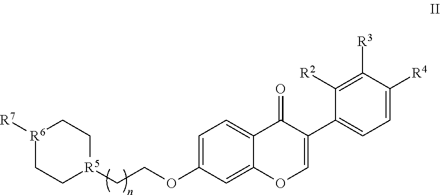
wherein each of R⁵ and R⁶ is independently selected from the group consisting of N, S, and O; and
wherein R⁷ is selected from the group consisting of H and hydroxyalkyl.

4. The compound of claim 1, wherein R¹ comprises a five-membered heterocycloalkyl.

5. The compound of claim 4, wherein the compound has a structure according to Formula III: 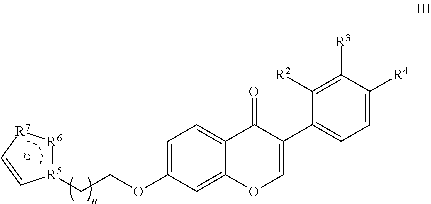
wherein each of R⁵, R⁶, and R⁷ is independently selected from the group consisting of C and N.

6. The compound of claim 1, wherein the compound has a structure according to Formula IV: 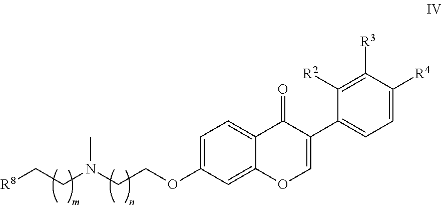
wherein m is from 1 to 10, and wherein R⁸ is OH, N(alkyl)₂, aromatic ring, piperidine or analogs thereof, morpholine, or analogs thereof, or piperazine or analogs thereof.

7. The compound of claim 1, having a structure selected from the group consisting of:

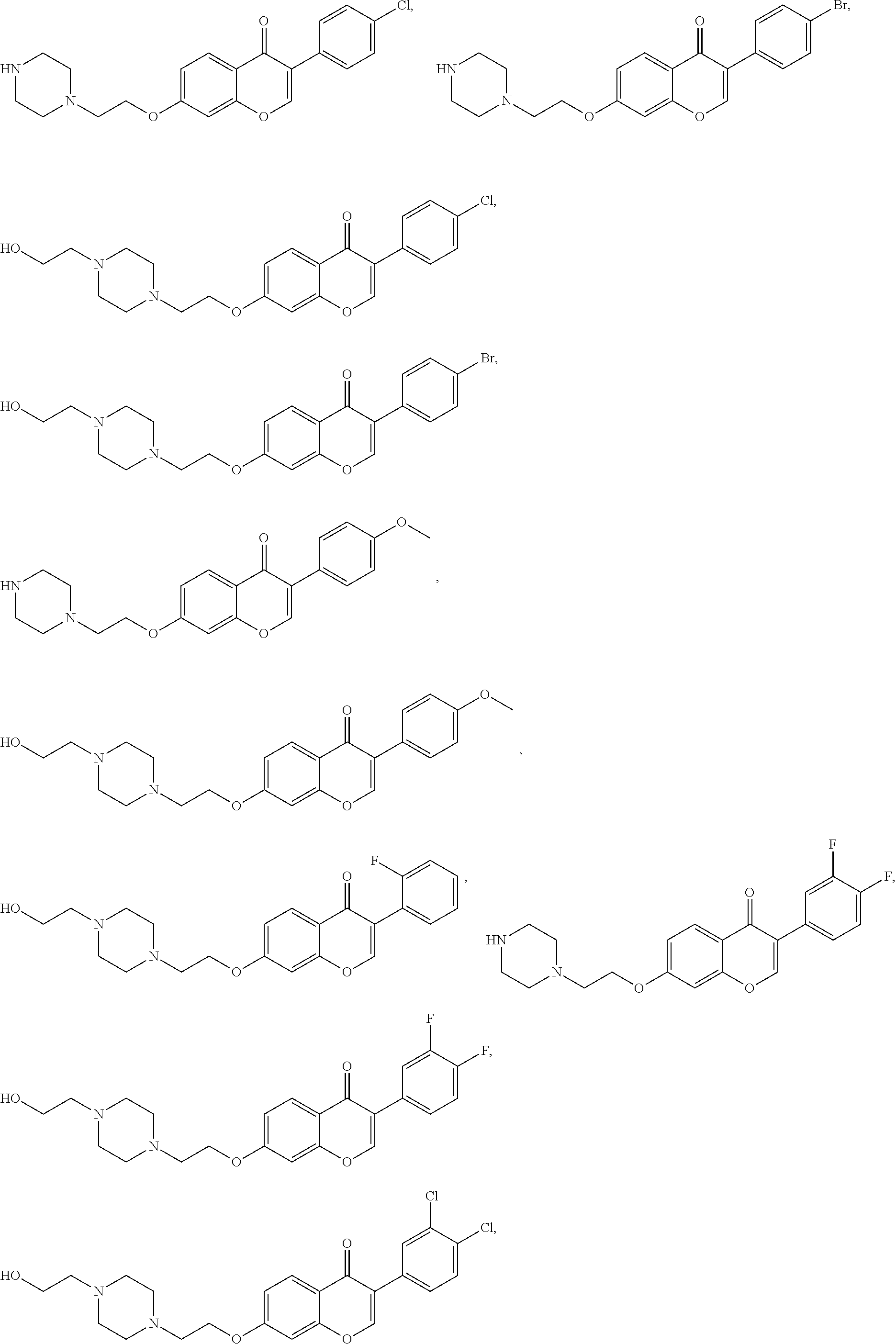

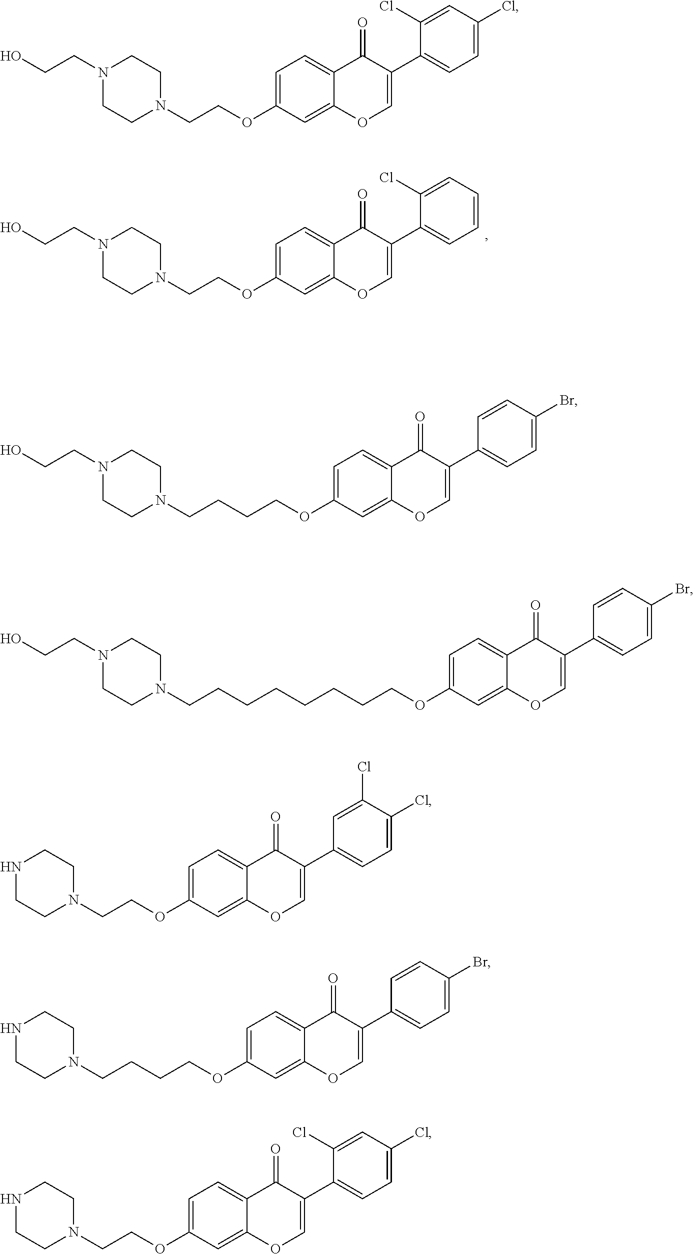

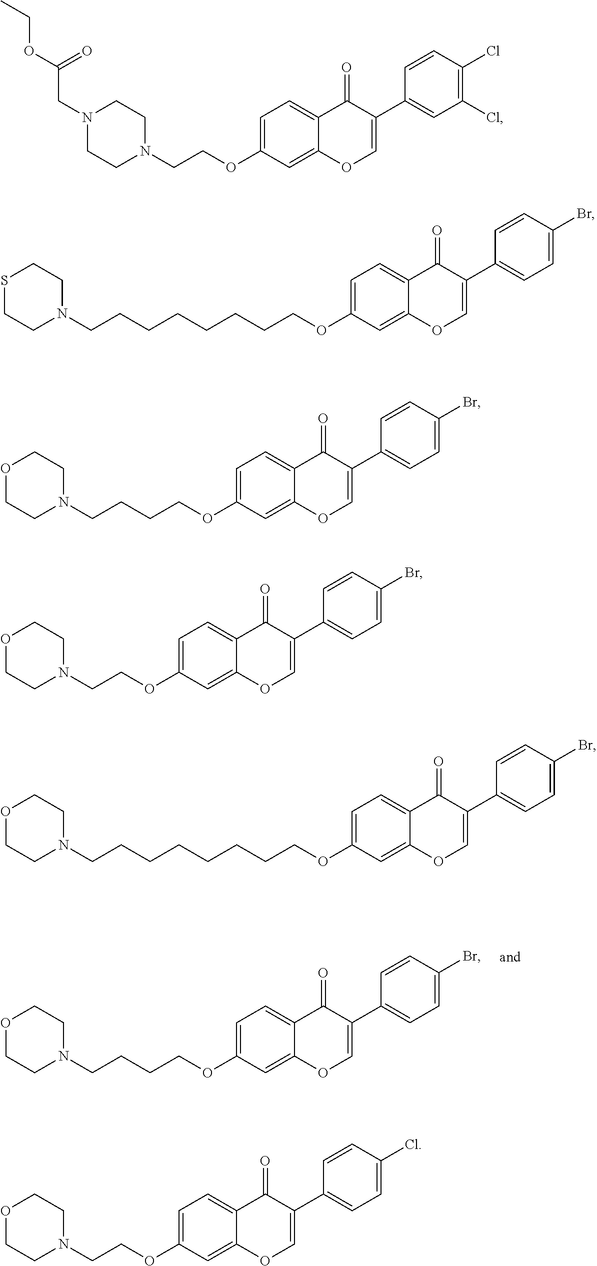

8. The compound of claim 1, having a structure selected from the group consisting of:

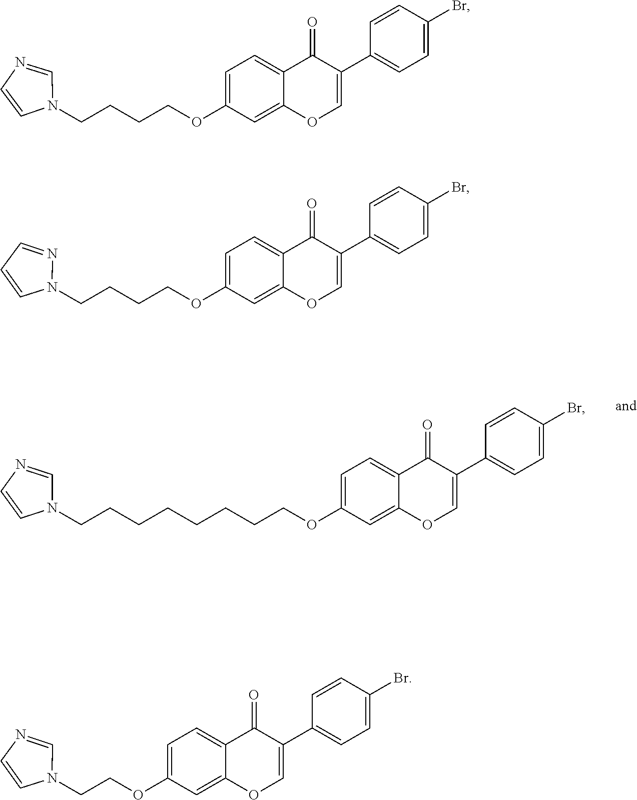 [SMILES: O=c1c(-c2ccc(Br)cc2)coc2cc(OCCCCn3ccnc3)ccc12 ; O=c1c(-c2ccc(Br)cc2)coc2cc(OCCCCn3cccn3)ccc12 ; O=c1c(-c2ccc(Br)cc2)coc2cc(OCCCCCCCCn3ccnc3)ccc12 ; O=c1c(-c2ccc(Br)cc2)coc2cc(OCCn3ccnc3)ccc12]

9. The compound of claim 1, having a structure selected from the group consisting of:

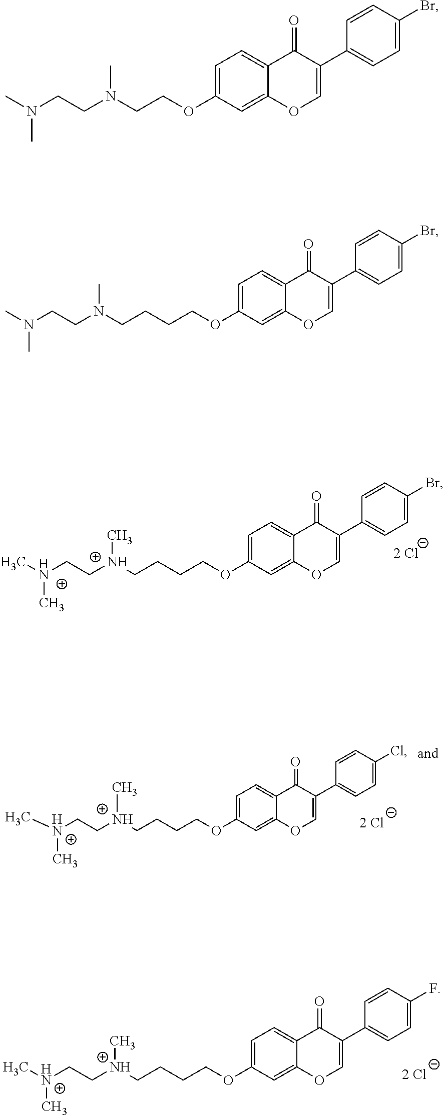

10. A method of treating a cancer, the method comprising administering one or more compounds according to claim 1 to a subject in need thereof.

11. A method of providing immunotherapy, treatment for diabetes, or treatment for obesity, the method comprising administering one or more compounds according to claim 1 to a subject in need thereof.
